(19) United States
(12) Patent Application Publication
(10) Pub. No.: US 2012/0177693 A1
(43) Pub. Date: Jul. 12, 2012

(54) DEEP LUNG PULMONARY DELIVERY OF TREPROSTINIL

(75) Inventors: David C. Cipolla, San Ramon, CA (US); Igor Gonda, San Francisco, CA (US); Babatunde Otulana, Danville, CA (US); Richard Morishige, Castro Valley, CA (US); Paul R. Bruinenberg, Livermore, CA (US)

(73) Assignee: Aradigm Corporation

(21) Appl. No.: 13/120,015

(22) PCT Filed: Sep. 24, 2009
(86) PCT No.: PCT/US09/58217

§ 371 Date: Jun. 2, 2011

Related U.S. Application Data
(60) Provisional application No. 61/100,017, filed on Sep. 25, 2008.

(51) Int. Cl. A61K 31/192; A61P 9/12; A61P 11/00; A61K 9/12

(52) U.S. Cl. 424/400; 514/569

(57) ABSTRACT

Administration of aerosolized Treprostinil formulations may provide a more homogeneous lung deposition of treprostinil, whereby making deep lung delivery possible.

### CROSS REFERENCE TO RELATED APPLICATIONS

The present application claims priority to U.S. provisional patent application No. 61/100,017 filed Sep. 25, 2009, which is incorporated herein by reference in its entirety.

### FIELD

The present application relates in general to therapeutic methods and in particular to therapeutic methods, which may involve pulmonary delivery of inhaled compounds. Such pulmonary delivery may reduce a dose, a side effect profile and/or a frequency of administration. In addition, such delivery may provide a depot effect in the peripheral lung with associated prolonged release into the systemic circulation.

### BACKGROUND

A large number of drugs may be generally administered by some type of injection. Although injecting drugs can provide a number of advantages, at times, for some patients, it may be inconvenient, and/or painful. One class of drugs generally administered by injection is prostacyclin and its analogues, such as Treprostinil.

Treprostinil is a synthetic analogue of prostacyclin. Treprostinil is marketed as Remodulin. As an analogue of protacyclin PGI2, treprostinil may affect vasodilation, which in turn may lower the blood pressure. Treprostinil may also inhibit platelet aggregation, though the role this phenomenon may play in relation to pulmonary hypertension has yet to be determined.

Treprostinil was first described in U.S. Pat. No. 4,306,075. U.S. Pat. No. 5,153,222 discloses use of treprostinil for treatment of pulmonary hypertension. U.S. Pat. No. 5,234,953 discloses treatment of congestive heart failure with treprostinil. U.S. Pat. Nos. 6,765,117 and 6,809,223 disclose stereoselective process for treprostinil synthesis. U.S. Pat. Nos. 6,521,212 and 6,756,033 describe administration of treprostinil by inhalation for treatment of pulmonary hypertension, peripheral vascular disease and other diseases and conditions. U.S. Pat. No. 6,054,486 discloses treatment of peripheral vascular disease with Treprostinil. U.S. Pat. No. 6,803,386 discloses administration of treprostinil for treating cancer, such as lung, liver, brain, pancreatic, kidney, prostate, breast, colon and head-neck cancer. US patent application publication no. 2005/0165111 discloses treprostinil treatment of ischemic lesions. U.S. Pat. No. 7,199,157 discloses that treprostinil treatment improves kidney functions. US patent application publication no. 2005/0282903 discloses treprostinil treatment of diabetic neuropathic foot ulcers. US patent application publication no. 2008/0280986 discloses treatment of interstitial lung disease with Treprostinil. US patent application publication no. 2008/0200449 discloses administration of Treprostinil via a metered dose inhaler. US patent application publication no. 2009/0163738 discloses an alternative process for preparation treprostinil. U.S. Pat. Nos. 7,417,070; 7,384,978 and 7,544,713 disclose oral forms of treprostinil. US patent application publication no. 2009/0036465 discloses administration of treprostinil in combination with Rho-kinase inhibitors. U.S. provisional application No. 61/176,268 discloses solid formulations of treprostinil.

Treprostinil may be used in the treatment and/or prevention of/for: pulmonary hypertension, ischemic diseases (e.g. peripheral vascular disease including peripheral arterial disease, Raynaud's phenomenon including Raynaud's disease and Raynaud's syndrome, Scleroderma including systemic sclerosis, myocardial ischemia, ischemic stroke, renal insufficiency), ischemic ulcers including digital ulcers, heart failure (including congestive heart failure), conditions requiring anticoagulation (e.g., post MI, post cardiac surgery), thrombotic microangiopathy, extracorporeal circulation, central retinal vein occlusion, atherosclerosis, inflammatory diseases (e.g., COPD, psoriasis), hypertension (e.g., preeclampsia), reproduction and parturition, cancer or other conditions of unregulated cell growth, cell/tissue preservation and other emerging therapeutic areas where prostacyclin treatment appears to have a beneficial role.

Treprostinil may be administered via a small infusion pump that a patient must wear at all times. Treprostinil may be given subcutaneously using an infusion set, or intravenously via acentral venous catheter if the patient is unable to tolerate the potential pain and discomfort of subcutaneous administration.

Treprostinil, under the trademark Remodulin, may be supplied in 20 mL vials, ranging in concentrations of 1 mg/mL, 2.5 mg/ML, 5 mg/mL, and 10 mg/mL. Treprostinil can be administered subcutaneously as supplied. For intravenous infusion, treprostinil is usually diluted with either sterile water or a 0.9% sodium chloride solution prior to administration.

The infusion rate may be normally initiated at 1.25 ng/kg/min for new patients, but may be reduced to 0.625 ng/kg/min if the normal rate provokes unwanted side effects in the patient. The infusion rate of treprostinil may be increased no more than 1.25 ng/kg/min per week for the first month, then no more than 2.5 ng/kg/min per week for the remaining duration of infusion. The infusion rate should ideally be high enough to improve symptoms of pulmonary hypertension, while minimizing unpleasant side effects.

A high percentage of patients report pain or other reaction at the infusion site. Other side effects may include headache, diarrhea, nausea, rash, jaw pain, vasodilation, dizziness, edema (swelling), pruritus (itching), and hypotension.

Remodulin® (treprostinil sodium) Injection can be a sterile sodium salt formulation for subcutaneous or intravenous administration. Remodulin can be supplied in 20 mL multi-use vials in four strengths, containing 1 mg/mL, 2.5 mg/mL, 5 mg/mL or 10 mg/mL of treprostinil. Each mL also contains 5.3 mg sodium chloride (except for the 10 mg/mL strength which contains 4.0 mg sodium chloride), 3.0 mg metacresol, 6.3 mg sodium citrate, and water for injection. Sodium hydroxide and hydrochloric acid may be added to adjust pH between 6.0 and 7.2.

Treprostinil has a degree of stability at room temperature and neutral pH.

Treprostinil sodium is (1R,2R,3aS,9aS)-[[2,3,3a,4,9,9a-Hexahydro-2-hydroxyl-1-[(3S)-3-hydroxyoctyl]-1H-benz[f] inden-5-yl]oxy]acetic acid monosodium salt. Treprostinil sodium has a molecular weight of 412.49 and a molecular formulation of C23H33NaO5.

The structural formula of treprostinil sodium is:

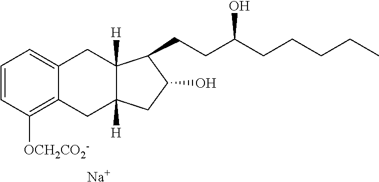

A potential problem with formulation drugs for pulmonary delivery may be that the formulation can include a relatively high concentration of the drug in order to reduce the volume so that the aerosolized volume can be readily inhaled by the patient. Another potential problem may be that upon delivery all of the drug in the formulation is immediately made available to the patient which can mean that too much drug may be made available too quickly. Further, it may be that the inhaled formulation does not provide any sustained release of drug over time. Formulations of the present invention endeavor to solve some or all of these problems.

### SUMMARY

In one embodiment, a method of treating or preventing a disease or condition, which is treatable or preventable with treprostinil, comprises administering by inhalation to a subject in need thereof, which may be a human, an aerosolized formulation comprising treprostinil or a pharmaceutically acceptable salt thereof and a carrier acceptable for pulmonary delivery, wherein said aerosolized formulation has an aerodynamic diameter of particles or droplets is no more than 10 microns or no more than 5 microns or in a range from 2 to 10 microns, and wherein said administering results in depositing the treprostinil in a deep lung, such that a ratio of central/peripheral lung deposits of the formulation is in a range of 1 to 2.0 or 1 to 1.9 or 1 to 1.8 or 1 to 1.7 or 1 to 1.6 or 1 to 1.5 or 1 to 1.45 or 1:1.4.

Diseases and conditions, which are treatable or preventable with treprostinil, include pulmonary hypertension, ischemic diseases (e.g. peripheral vascular disease including peripheral arterial disease, Raynaud's phenomenon including Raynaud's disease and Raynaud's syndrome, Scleroderma including systemic sclerosis, myocardial ischemia, ischemic stroke, renal insufficiency), ischemic ulcers including digital ulcers, diabetic neuropathic and neuroischemic ulcer, heart failure (including congestive heart failure), conditions requiring anticoagulation (e.g., post MI, post cardiac surgery), thrombotic microangiopathy, extracorporeal circulation, central retinal vein occlusion, atherosclerosis, inflammatory diseases (e.g., COPD, psoriasis), hypertension (e.g., preeclampsia), reproduction and parturition, cancer or other conditions of unregulated cell growth, cell/tissue preservation and other emerging therapeutic areas where prostacyclin treatment appears to have a beneficial role.

Physiologically acceptable salts of Treprostinil include salts derived from bases. Base salts include ammonium salts (such as quaternary ammonium salts), alkali metal salts such as those of sodium and potassium, alkaline earth metal salts such as those of calcium and magnesium, salts with organic bases such as dicyclohexylamine and N-methyl-D-glucamine, and salts with amino acids such as arginine and lysine.

Quaternary ammonium salts can be formed, for example, by reaction with lower alkyl halides, such as methyl, ethyl, propyl, and butyl chlorides, bromides, and iodides, with dialkyl sulphates, with long chain halides, such as decyl, lauryl, myristyl, and stearyl chlorides, bromides, and iodides, and with aralkyl halides, such as benzyl and phenethyl bromides.

The carrier(s) must be “acceptable” in the sense of being compatible with the other ingredients of the formulation and not deleterious to the recipient thereof. The carrier may be a liquid or a solid.

Aerosolized delivery of Treprostinil may result in a more homogeneous distribution of treprostinil in a lung, so that deep lung delivery is obtained. The deep lung delivery may result in an increased T_{MAX} and a decreased C_{MAX} as compared to upper respiratory tract delivery.

In some embodiments, the formulation may be a liposome free formulation. Yet in some embodiments, trepostinil may be administered together with liposomes.

Using polymer coatings or liposomes with the treprostinil may further increase The T_{MAX} may increased further and further decrease the C_{MAX}. The decreased C_{MAX} may result in reduced side effects, and the increased T_{MAX} results in a more convenient delivery.

This invention may relate to inhaled delivery of drugs which may exhibit delayed absorption from the peripheral lung or alveolar space due to sequestering in the lung interstitium, binding to cells, membranes or receptors, uptake by alveolar cells or macrophages, or via some other mechanism. Of particular interest are drugs which have systemic side effects and/or which exhibit pharmacological activity in the deep lung or alveolar space; e.g., treprostinil.

The methodology of the present invention provides increased efficacy at lower doses due to the sustained presence of the drug at the site of action in the deep lung.

The invention also provides a reduction in side effects resulting from a decreased C_{MAX} as well as a prolongation of T_{MAX} in the systemic circulation.

There may be multiple ways to enable and optimize delivery of the aforementioned drugs to the deep lung. For example, aerosol delivery system include DPIs, MDIs, nebulizers, solution inhalers, vapor condensation aerosol generators. Delivery can also be obtained via the use of aerosols containing lower density or geometrically smaller droplets or particles, or via slower inhalation flow rates to reduce impaction in the oropharynx and central airways.

Of particular interest is the use of Aradigm's AERx Essence system and AERx family of devices, which are described, for example, in U.S. Pat. Nos. 5,497,763; and 6,123,068 and related U.S. and non-U.S. patents and publications all of which are incorporated herein by reference to disclose and describe delivery devices, packets that hold drug and methods of administration. In the present human PK and gamma scintigraphic clinical trial, the AERx Essence system and the Nebu-Tec OPTINEB nebulizer were compared in a cross over fashion in 14 healthy subjects using inhaled treprostinil sodium. The AERx system provided greater deep lung delivery (mean Central/Peripheral lung ratio from planar gamma scintigraphy of 1.39) as compared to the nebulizer (mean Central/Peripheral lung (C/P) ratio of 3.96) which was associated with a delayed T_{MAX} for the AERx Essence System (mean 21 minutes) than for that of the nebulizer (mean 9 minutes). The C_{MAX} was also lower for AERx (mean 0.64 ng/mL) than for the nebulizer (mean 0.762 ng/mL) even with a 20% greater treprostinil lung dose for AERx than for the nebulizer, suggesting that adverse events may be reduced for an AERx Essence inhalation product.

Generally, adverse events are related to the peak concentration of treprostinil in the blood stream (Voswinckel et al., “Favorable Effects of Inhaled Treprostinil in Severe Pulmonary Hypertension: Results from Randomized Controlled Pilot Studies” J. Am. Coll. Cardiol., 48(8):1672-1681 (2006)) and the authors suggest, “that the systemic plasma concentration might determine the systemic side effect profile, while local lung tissue concentrations determine the pulmonary vasodilator effect.”

Voswinckel et al. compare and contrast inhaled iloprost to inhaled treprostinil and state the following:

“The long duration of pulmonary vasodilation after a single inhalation of treprostinil may be partially explained by the stability of this prostanoid. We speculate that treprostinil is stored in the lung tissue after inhalation, providing a slow release from the alveolar lining layer or the interstitial compartment to the pulmonary vascular smooth muscle cells. Peak plasma concentrations of treprostinil were observed 10 to 15 min after inhalation. This is considerably later compared to inhaled iloprost, with which peak plasma levels were found immediately after the completion of the inhalation session and plasma half-life was only about 8 min. This might explain the slower rate of onset of the pulmonary vasodilator effects and the virtual absence of systemic side effects despite the administration of higher doses of treprostinil. Similar to inhaled iloprost, the duration of the hemodynamic effect of treprostinil outlasted the plasma concentrations . . . . It is also possible that differences in binding characteristics to prostaglandin-E receptors and prostaglandin-I receptors contribute to the different pharmacodynamic profiles of inhaled treprostinil versus iloprost. Prostanoids and their analogs selectively bind to their 7 cognate prostanoid receptors, which initiate second messenger signaling that leads to either vasodilation or vasoconstriction, depending on the prostanoid receptor specificity of the analog and the receptor distribution in the respective vascular bed. Differences between treprostinil and iloprost in prostanoid receptor specificity and activation, together with tissue binding characteristics, may explain the improved pulmonary selectivity of inhaled treprostinil . . . .”

In the above description, the authors suggest many possible explanations for why treprostinil and iloprost differ in their absorption and side effect profiles, primarily due to factors specific to the drug; e.g. differences in the individual drug stability profile and/or drug structures that effect the disposition in the lung and body. Both drugs were considered efficacious. However, these authors failed to anticipate that the mode of inhalation could improve the drug's pharmacokinetic, pharmacodynamic and side effect profile. In our clinical studies, by depositing the treprostinil more consistently and deeper in the lung; e.g. using the AERx System, the peak plasma concentration was further delayed by a factor of two over that for the nebulizer. There was one subject in the nebulizer arm who exhibited a delayed Tmax of ˜20 minutes and the gamma scintigraphic image showed a C/P ratio of 1.5, indicating peripheral lung deposition, unlike the typical nebulizer image. This finding corroborates the association of deep lung penetration with slower absorption into the systemic circulation. The achievement of deeper lung penetration (and the associated delayed systemic uptake) in one subject in the nebulizer arm is not due to a difference in the nebulizer aerosol particle size distribution for that subject, but is likely due to differences in the inhalation maneuver, or the airway or lung geometry.

This invention can be enhanced by the use of specific formulation agents or in combination with other delivery strategies. For example, a variety of formulations, polymers, gels, emulsions, particulates or suspensions, either singly or in combination, could be used to increase the sustained release profile in the deep lung and enhance the delay in systemic absorption. The rate of release can be designed to provide dosing over a period of hours, days or weeks. This can be accomplished in many ways; e.g., by coating the aerosol particles with excipients that dissolve slowly in the aqueous environment of the lung (e.g., PLGA, polymers, etc.) or by coating or encapsulating the drug molecules with excipients that release the drug slowly (e.g., liposomes, surfactants, etc.). Other formulation strategies also exist for delaying or extending the release profile of the drug in the lung. Even though the same amount of drug may still be delivered to the lung in these scenarios, the peak drug concentration that is absorbed into the bloodstream after inhalation would be attenuated resulting in a reduction in, or elimination of, the side effect profile. A potential additional feature of this delivery modality is one of convenience for the patient. The frequency of dosing may also be reduced, thereby potentially increasing patient convenience or compliance to therapy, and thus efficacy.

Although so far we have discussed only treatment of PAH patients with treprostinil, there is no intention to limit the application of this intellectual property to treatment of PAH patients nor to limit the choice of drug to treprostinil. In fact, there are many patients and indications for which this therapeutic improvement may be beneficial, including lung cancer, cystic fibrosis, bronchiectasis, pneumonia, COPD, asthma, pulmonary fibrosis, and other lung diseases. There are also many potential drugs which may benefit from this invention including various antibiotics such as penicillin, cephalosporin, fluoroquinolone, tetracycline, or macrolide.

These and other objects, advantages, and features of the invention will become apparent to those persons skilled in the art upon reading the details of the formulations, methods and devices as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a block diagram showing the disposition of the subjects.

FIG. 2 is a graph showing the mean plasma drug concentrations.

FIG. 3 is a graph showing mean plasma drug concentrations.

FIG. 4 is a table summarizing demographic data.

FIG. 5 is a table showing a summary of recovery of labeled drug.

FIG. 6 is a table showing a summary of recovery of percent emitted radiolabeled drug.

FIG. 7 is a table showing a summary of recovery of radiolabeled drug delivered via AERx.

FIG. 8 is a summary of recovery of radiolabeled drug delivered by nebulizer.

FIG. 9 is a summary of derivation of lung dose of drug delivered by AERx.

FIG. 10 is a summary of derivation of lung dose of drug delivered by nebulizer.

FIG. 11 is a summary of individual drug pharmacokinetic parameters.

FIG. 12 is a table summarizing individual drug dose adjustment pharmacokinetics.

FIG. 13 is a table showing a summary of adverse events.

FIG. 14 is a second table showing a summary of adverse events.

FIG. 15 is a third table showing a summary of adverse events.

FIG. 16 is a table showing abnormal laboratory value listings for each subject.

FIG. 17 is a table showing hematology out of range results.

FIG. 18 is a table showing urinalysis out of range results.

FIGS. 19A-H are tables each of which show summaries of lung function test results.

### DEFINITIONS

C_{MAX} is the maximum concentration of a drug in the body after dosing.

T_{MAX} is the period of time after dosing that it takes for C_{MAX} to occur.

Abbreviations used in the text:

### AE Adverse Event

### ALP Alkaline Phosphatase

### ALT Alanine Aminotransferase

### ANOVA Analysis of Variance

### AST Aspartate Aminotransferase

### ARSAC Administration of Radioactive Substances Advisory Committee

### ATS American Thoracic Society

AUC Area Under the (concentration-time) Curve

### BID Twice Daily

### BMI Body Mass Index

### BP Blood Pressure

### BUN Blood Urea Nitrogen

### C/P Central-to-Peripheral Ratio

cGMP Current Good Manufacturing Practices

### CI Confidence Interval

Cmax Maximum plasma drug concentration

### CPK Creatinine Phosphokinase

### CRA Clinical Research Associate

### CRF Case Report Form

DPS Disintegration per second

### ECG Electrocardiogram

### ERS European Respiratory Society

FEF_{25-75%} Forced Expiratory Flow between 25-75% of FVC
FEV₁ Forced expiratory volume in 1 second

### FVC Forced Vital Capacity

### GCP Good Clinical Practice Guidelines

GMc Geometric Mean, corrected
HbA1c Glycosylated hemoglobin
HBV Hepatitis B virus

### Hct Hematocrit

### hCG Human Choriogonadotropin

HCV Hepatitis C virus

### HEENT Head, Eyes, Ears, Nose and Throat

### HR Heart Rate

### HREC Human Research Ethics Committee (IRB)

### Hgb Hemoglobin

HIV Human Immunodeficiency virus

### ICF Information and Consent Form/s

### IB Investigator Brochure

### ICH International Conference on Harmonization

### IND Investigational New Drug

### INR International Normalized Ratio

### IDMB Independent Data Monitoring Board

### IRB Institutional Review Board

### ITT Intent-to-Treat

Kel Elimination rate constant

### MBq Mega Becquerel

### LOQ Level of Quantification

### MCH Mean Corpuscular Hemoglobin

### MCV Mean Corpuscular Volume

### MCHC Mean Corpuscular Hemoglobin Concentration

### mSv Milli-Sievert

### PD Pharmacodynamic

### PEFR Peak Expiratory Flow Rate

### PI Principal Investigator

### PK Pharmacokinetic

pKa Negative log of the acid dissociation constant, Ka

### QA Quality Assurance

### QC Quality Control

### RBC Red Blood Cell

### ROI Region of Interest

### RR Respiratory Rate

### SAE Serious Adverse Event

### SOP Standard Operating Procedures

### SD Standard Deviation

### SV Sievert

^{99m}Tc-DTPA Technetium-labeled diethylenetriamine pentaacetate
Tmax Time to maximum plasma drug concentration

### WBC White Blood Cell

Before the present formulations, methods and devices are described, it is to be understood that this invention is not limited to particular formulations, methods and devices described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a drug” includes a plurality of such drugs and reference to “the particle” includes reference to one or more particles and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### 9. Investigational Plan

### 9.1 Overall Study Design and Plan

This was an open label study conducted in a single-center, using a randomized, two-way crossover design. Sixteen healthy adult male subjects were to be enrolled to receive study treatments. Upon provision of written informed consent, each study candidate underwent a pre-study evaluation and screening to determine eligibility to participate.

Subjects received instruction and training in the proper use of the Nebu-Tec Optineb nebulizer and AERx Essence System using drug-free dosage forms.

On each of two dosing days, eligible subjects underwent dosing with ^{99m}Tc-labeled treprostinil sodium using either the AERx Essence System or the Nebu-Tec Optineb nebulizer. Following their initial study dose, subjects underwent a washout period of approximately 48 hours before completing a second (crossover) study dose.

Immediately following each study dose, subjects underwent gamma scintigraphy and multiple samplings of venous blood to characterize treprostinil lung deposition and plasma pharmacokinetics.

| Day 1 | Day 2 | Day 3 | Day 4 | Day 5 |
|---|---|---|---|---|
| Admit AB | Dose AB | Dose CD | Crossover | Crossover |
| | | | Dose AB | Dose CD |
| | Discharge | Discharge | Discharge | Discharge |
| | AB | CD | AB | CD |
| | Admit CD | Admit AB | Admit CD | |

Treatment Group A = AERx Essence → Nebu-Tec Optineb (n = 4)
Treatment Group B = Nebu-Tec Optineb → AERx Essence (n = 4)
Treatment Group C = AERx Essence → Nebu-Tec Optineb (n = 4)
Treatment Group D = Nebu-Tec Optineb → AERx Essence (n = 4)

Subjects also underwent a Krypton-81m (^{81m}Kr) gas ventilation imaging procedure. This procedure could have been performed on any dosing day followed by a 30 minute washout before any of the aerosol dosing procedures, if there were logistical/scheduling problems, ventilation imaging could have been done after dosing. Alternatively the ^{81m}Kr ventilation scan could have been performed on a separate visit. Additionally transmission images were also acquired this could have been performed on any dosing day prior to any of the aerosol dosing procedures or on a separate visit

Subjects received each of the following treatments according to a randomization code produced by Simbec Research using the PROC PLAN procedure of SAS Version 9.1.3.

There were at least 48 hours between dose administrations.

Each study period was of 1 day's duration.

The study took place in the Clinical Centre of Simbec Research under full medical and nursing supervision.

### 9.2 Discussion of Study Design, Including the Choice of Control Groups

The primary objective of the trial was to compare the emitted dose, delivered lung dose, and the central-to-peripheral (sC/P) lung deposition of radiolabeled treprostinil sodium delivered via the AERx Essence System versus the Nebu-Tec Optineb nebulizer using gamma scintigraphy. The secondary objectives were to compare the venous plasma pharmacokinetic profile for treprostinil delivered via the AERx Essence System to the Nebu-Tec Optineb nebulizer, assess the safety and tolerability of inhaled treprostinil sodium via both test devices, compare the percent dose (emitted and loaded) of radiolabeled treprostinil sodium in the oropharyngeal region from both devices and compare the percent loaded dose remaining in both devices and associated equipment where appropriate e.g. mouthpiece, exhalation filter, tubing.

The trial was the second time treprostinil sodium for inhalation had been given to healthy volunteers via the AERx Essence System, and therefore was the basis for the future development of treprostinil sodium for inhalation. Data on the safety and tolerability of treprostinil sodium for inhalation and on the appropriateness of its pharmacokinetics for human use were obtained.

### 9.3 Selection of Study Population

### 9.3.1 Inclusion Criteria

Healthy male subjects aged 18 to 55 years, inclusive at the time of first dose.
Subjects must be willing to use an acceptable method of birth control during the study administration period and at least 30 days afterward, e.g.
   Oral contraceptive+condom
   Intra-uterine device (IUD)+condom
   Diaphragm with spermacide+condom
Normal spirometry (FVC and FEV₁≧80% predicted for age, height, sex; PEFR≧80% predicted for age, height, sex; FEV₁/FVC≧0.7).
Systolic blood pressure of >100 mm Hg and diastolic blood pressure of >60 mm Hg.
Non-smoker for at least 12 months prior to screening visit
No clinically significant abnormal serum, biochemistry, hematology and urine examination values within 14 days of dosing in period 1.
12-Lead ECG showing no clinically significant abnormality
Negative urine test for alcohol and drugs of abuse at screening.
Negative blood test for hepatitis B surface antigens, hepatitis C antibodies and HIV
BMI within the range of 20 to 33, inclusive
Height ≧152 cm (60 inches)
Fluency in written and spoken English language
Ability to use the AERx Essence System per Sponsor's instructions
Ability to use the Nebu-tec Optineb nebulizer per Sponsor's instructions
Ability to provide written informed consent.

### 9.3.2 Exclusion Criteria

Evidence of clinically significant cardiovascular, hematological, hepatic, renal, neurological, or psychiatric disease, including but not limited to:
   Myocardial infarction, coronary bypass surgery, or angioplasty within the past 12 months
   Congestive heart failure requiring hospitalization within the past 12 months
   Uncontrolled arrhythmias
   Transient ischemic attacks
   History of multiple sclerosis
   Seizures within the past 10 years or taking seizure medication
Evidence of clinically significant laboratory test results, including but not limited to:
   Elevated AST (SGOT), ALT (SGPT), ALP, bilirubin or creatinine
   White blood cell count or platelet count at a level considered to be clinically significant.
   Hematocrit above or below a level considered to be clinically significant.
History of asthma or chronic obstructive pulmonary disease within 5 years. This includes subjects who require routine treatment with oral or inhaled corticosteroids or bronchodilators
History of upper respiratory tract infection within 14 days prior to the first dose in Period 1
Known or suspected allergy to treprostinil sodium or any excipients of the formulation.
History of orthostatic hypotension.
Known or suspected allergy to ^{99m}Tc-DTPA
Participation in a study in which radioisotopes were administered within 12 months preceding the first dose of Period 1 of this study, or has been exposed to radiation excess within the last 12 months (e.g., x-rays, handling of radiolabel materials). Radiation excess is determined on a case-by-case basis following review by the Principal Investigator or designee.
Participation in a study of a New Chemical Entity (NCE) within 4 months or a marketed drug within 3 months preceding the first dose in Period 1.
Subjects who, within 14 days preceding the first dose in Period 1, have taken any prescription or non-prescription medication that the Principal Investigator or designee considers would interfere with the study outcome.
Subjects who have consumed more than 2 units of alcohol per day from the seven (7) days prior to the administration of the first dose or who have consumed any alcohol within the 48 hour period prior to the first dose.
Subjects who, in the opinion of the Investigator, are not suitable candidates for enrolment or would not comply with the requirements for the trial.
Any other condition which, in the Investigator's opinion, contraindicates study participation.
   9.3.3 Removal of Subjects from Therapy or Assessment

Each subject was informed of his/her right to withdraw from the study at any time and for any reason.

The investigator was able to withdraw a subject from the study at any time if he/she considered that the subject's health was compromised by remaining in the study or the subject was not sufficiently cooperative.

### 9.4 Treatments

### 9.4.1 Treatments Administered

The study drug was treprostinil sodium for inhalation in a formulation containing ^{99m}Tc-DTPA. Aradigm (via Lung Rx) provided the “bulk” treprostinil sodium that was used in this study.

A single “bulk” treprostinil sodium formulation (600 μg/mL) was used for both the Nebu-Tec Optineb nebulizer and the AERx Essence. The bulk drug solutions were diluted by the addition of ^{99m}Tc-DTPA (2000 MBq/mL) at a ratio of 19:1, i.e. 0.05 mL of ^{99m}Tc-DTPA was added to 0.950 mL of bulk drug solution. Each mL of the radiolabeled drug solutions therefore contained 100 MBq of ^{99m}Tc-DTPA and 570 μg of treprostinil. The radiolabel ^{99m}Tc as ^{99m}Tc-DTPA was acquired from an approved supplier (i.e., the Medical Physics Department, University Hospital of Wales, Heath, Cardiff [Manufacturers License Number: MS/IMP18523]).

The Optineb nebulizer cup was filled with 2 mL of radiolabeled treprostinil sodium formulation, resulting in a nebulizer loaded treprostinil dose of 1140 μg. Assuming that the Optineb delivers a treprostinil lung dose of approximately 4.75 μg per inhalation, the total estimated treprostinil lung dose delivered over the 6 inhalation study dose was 28.5 μg.

The AERx formulation had the same concentrations of excipients as the nebulizer solution. For the AERx Essence System, the treprostinil sodium formulation was 570 μg/mL. AERx dosage form strips had a volume of 0.050 mL, resulting in a loaded treprostinil dose of 28.5 μg. The AERx Essence study dose consisted of 2 inhalations, and assuming a treprostinil lung dose of approximately 13 μg per inhalation, a total treprostinil lung dose of approximately 26 μg was delivered. Drug administration was documented in the Case Report Forms and on the Simbec Drug Administration Records.

There were at least 48 hours between doses.

### 9.4.2 Identity of Investigational Product(s)

### 9.4.2.1 Study Drug

The study drug was treprostinil sodium for inhalation in a formulation containing ^{99m}Tc-DTPA. Aradigm (via Lung Rx) will provide the “bulk” treprostinil sodium used in this study.

### 9.4.2.2 Radiolabeling Procedure and Formulations

The radiolabeling process followed established methods used by Aradigm Corporation in a number of previous studies^{5,6}. A solution of the gamma-emitting radiopharmaceutical, ^{99m}Tc-DTPA (radioisotope t½=6 h), was added to each treprostinil sodium formulation to quantify the deposition of the aerosolized product.

For both devices the specific activity per microgram drug was 0.18 MBq/μg.

The ^{99m}TcDTPA activity in the 50 μL AERx dosage form was 5 MBq. This number was based upon the addition of not more than 5% (v/v) of a 2000 MBq/mL ^{99m}Tc-DTPA solution. For a delivery efficiency of 50%, the AERx Essence System will then deliver 5 MBq to the lungs i.e. 2×2.5 MBq.

The Optineb nebulizer cup was filled with 2 mL of the ^{99m}Tc-DTPA treprostinil solution, i.e. 200 MBq ^{99m}Tc-DTPA and 1140 μg of drug. Each emitted dose (inhalation) from the nebulizer delivered 11 μL, i.e., 1.1 MBq ^{99m}Tc-DTPA and 6 inhalation were administered for each dose. Since only 76% of the emitted dose was expected to reach the lungs, approximately 5.0 MBq ^{99m}Tc-DTPA was deposited in the lungs.

Prior to the clinical study, the integrity of treprostinil sodium and the surrogate radiolabel was tested in vitro using appropriate assays (treprostinil sodium by SEC and IEC HPLC; ^{99m}Tc by gamma camera and gamma counting). The emitted dose and particle size distribution of the aerosols in vitro was evaluated for each delivery system using treprostinil sodium and ^{99m}Tc-DTPA to ascertain that the label followed the active compound with high fidelity. In addition, confirmatory experiments were conducted to demonstrate that the quantity and quality of emitted treprostinil sodium aerosol were the same for the labeled and non-labeled formulations (the amount of ^{99m}Tc-DTPA incorporated into the formulation was minimal and not greater than 5% v/v of the treprostinil sodium formulation). Following in vitro radiolabeling validation studies, Master Batch Records were created to allow a mixture of ^{99m}Tc-DTPA with the treprostinil sodium formulation and manual filling of AERx dosage forms at Simbec Research Ltd. on each dosing day.

### 9.4.2.3 Radiation Dosimetry

The maximum radiation dose received by the subjects was 0.254 milli-Sieverts (mSv) for both aerosol exposures and the ^{81m}Kr inhalation, this is equivalent to 2 months background radiation exposure. The radiation exposure to the subjects was expressed in terms of the effective dose (ED). This is a single figure specifying a hypothetical uniform whole body dose equivalent that would involve the same risk as the actual (non-uniform) dose distribution.

The dose equivalent is expressed in units of Sieverts (Sv), and is a measure of the energy absorbed by biological tissues (i.e., Jkg⁻¹ (Gray)) and also takes into account a quality factor. In the case of gamma radiation, the quality factor is 1. Thus, the dose equivalent is equal to the absorbed dose. The effective dose equivalent is the sum of the weighted organ dose equivalents. The weighting factors⁷ reflect the different radiosensitivity of various organs and tissues.

In the current study, the calculations of ED were based upon data in the Notes for Guidance on the Administration of Radioactive Substances to Persons for Purposes of Diagnosis, Treatment or Research⁽³⁾ and the Annals of the International Commission on Radiological Protection (ICRP) 1998⁽⁸⁾. These documents provided information concerning the ED arising from a given maximum administered dose by a particular route of administration. The administered dose is defined in terms of MBq (i.e., 1 Becquerel=1 disintegration per second (DPS), 1 MBq=106 DPS). Thus, the ^{81m}Kr ventilation image ED (0.02 mSv) was derived from specific data relating to this diagnostic procedure. The ED for the 99mTc administration was extrapolated from data relating to lung ventilation imaging.

For comparison, the ED associated with common diagnostic x-ray and nuclear medicine procedures⁹ are as follows:

| | | Equivalent Period of Natural | |
|---|---|---|---|
| | ED (mSv) | Background Radiation | |
| Radiographic Test | | | |
| Barium enema | 7.69 | 3.8 | Years |
| Barium meal | 3.83 | 2 | Years |
| Thoracic spine | 0.92 | 6 | Months |
| Skull | 0.15 | 1 | Month |
| Chest | 0.05 | 10 | Days |
| Nuclear Medicine | | | |
| Bone scan | 2.15-3.83 | 1 to 2 | Years |
| Lung perfusion/Liver | 0.92-1.22 | 6 to 7 | Months |
| Current Study | | | |
| Radiolabel Deposition | 0.254 | Approximately 2 | Months |

### 9.4.2.4 Study Drug Inventory and Storage

The study medication was stored at Simbec Research facilities in a secure, dry environment, at room temperature (+15° to +30° C.).

The Principal Investigator was responsible for the dispensing, inventory and accountability of all drug supplies. An accurate record of the disposition of all drug supplies was maintained in a Drug Accountability Record. During the study or upon completion or termination of the study, the investigator will return all unused drug supplies and the Drug Accountability Record to Aradigm Corporation.

A record of the dates and quantity of medication dispensed to each subject on each dosing day was made in the subject's CRF.

### 9.4.4 Selection of Doses in the Study

The selection of doses within the study was based upon data from previous healthy volunteer studies with treprostinil sodium for inhalation.

The Optineb nebulizer cup was filled with 2 mL of radiolabeled treprostinil sodium formulation, resulting in a nebulizer loaded treprostinil dose of 1140 μg. Assuming that the Optineb delivers a treprostinil lung dose of approximately 4.75 μg per inhalation, the total estimated treprostinil lung dose delivered over the 6 inhalation study dose was 28.5 μg.

The AERx formulation had the same concentrations of excipients as the nebulizer solution. For the AERx Essence System, the treprostinil sodium formulation was 570 μg/mL. AERx dosage form strips had a volume of 0.050 mL, resulting in a loaded treprostinil dose of 28.5 μg. The AERx Essence study dose consisted of 2 inhalations, and assuming a treprostinil lung dose of approximately 13 μg per inhalation, a total treprostinil lung dose of approximately 26 μg was delivered.

Prior to the clinical study, the integrity of treprostinil sodium and the surrogate radiolabel were tested in vitro using appropriate assays (treprostinil sodium by SEC and IEC HPLC; ^{99m}Tc by gamma camera and gamma counting). The emitted dose and particle size distribution of the aerosols in vitro were evaluated for each delivery system using treprostinil sodium and ^{99m}Tc-DTPA to ascertain that the label follows the active compound with high fidelity.

### 9.4.5 Selection and Timing of Dose for Each Subject

Doses were administered at approximately 45 minute intervals starting at approximately 11:00 am. Due to the procedures post dose, dosing lasted for approximately 5 hours each day.

On dosing days, subjects received a light breakfast and a light lunch. Food was not consumed from 2 hours prior to dosing and 2 hours post dosing. Fluids were also withheld from 2 hours prior to dosing and 2 hours post dosing. Immediately following dosing, subjects rinsed their mouths with water, expelled the washings for collection, and swallowed a piece of bread.

### 9.4.6. Blinding

This was an open label study.

### 9.4.7 Prior and Concomitant Therapy

Any medication taken by subjects during the study was recorded on the CRF. Subjects were withdrawn from the study if medication was taken to treat exclusionary medical conditions as listed in Section 3.2.2 of the study protocol.

A short-acting β2 inhaler, was part of a standard emergency kit, and was available for use at all times in case of emergent bronchospasm.

Subjects who within 14 days preceding the first dose in Period 1, had taken any prescription or non-prescription medication that the Principal Investigator or designee considered would have interfered with the study outcome were excluded from the study.

### 9.4.8 Treatment Compliance

Doses were taken under supervision.

### 9.5 Efficacy and Safety Variables

### 9.5.1 Efficacy and Safety Measurements Assessed and Flow Chart

### 9.5.1.1 Efficacy

In this study, the radiolabel marker (^{99m}Tc-DTPA) deposition profiles obtained following administration of a radiolabeled treprostinil sodium formulation will be evaluated using gamma to assess the performance for the two delivery systems. Gamma scintigraphy offers a precise and accurate method of evaluating the deposition of an inhaled radiolabeled aerosol in the oropharynx and lung.

### 9.5.1.2 Safety Measurements

The safety end-points for this study included:
FEV₁, FVC and PEFR values
vital signs
ECGs
adverse events
safety laboratory results.

### 9.5.1.3 Pharmacodynamics

Not applicable.

### 9.5.1.4 Study Flowchart

| | | Assessment | | |
|---|---|---|---|---|
| | Screening | Period: | | Follow-up |
| | (Visit 1) | (Visits 2 & 3) | | (Visit 4) |
| | Study Day | | | |
| | | | | 1-5 days post |
| | | | | last study |
| | −14 to −2 | Day −1 | Day 1 | procedure |
| Confinement | | X | X | |
| Outpatient | X | | | X |
| Informed Consent and | X | | | |
| Medical History | | | | |
| Height and Weight | X | | | |
| Physical Examination | X | | | X |
| Vital signs | X | X | | X |
| Inclusion/Exclusion Criteria | X | X | | |
| 12-lead ECG | X | | | X |
| Laboratory Examination¹ | X | | | X |
| Urine Drug and alcohol | X | X | | |
| screen | | | | |
| Hepatitis B, HCV and HIV | X | | | |
| tests | | | | |
| Randomisation | | | X | |
| Study Drug Administration | | | X | |
| Previous and Concomitant | X | X | X | X |
| Medication | | | | |
| Pharmacokinetic Blood | | | X | |
| Sampling² | | | | |
| Adverse Events | | X | X | X |
| Spirometry³ | X | X | X | X |
| Inhalation technique with | X | | | |
| AERx essence system | | | | |
| KR scan | X* | X* | X* | |
| Training with devices | | | X | |

¹Biochemistry, Hematology and Urinalysis.
²Pharmacokinetic blood sampling occurred on Day 1 of each period at approximately 1 hour prior to dosing and at 2, 3, 5, 7, 10, 15, 20, 30, 60, 90, 120, 180, 240, 300 and 360 minutes after each study dose.
³Spirometry measurements (FVC, FEV, & PEFR), were taken after imaging procedures, and approximately 65 minutes and 4 hours post dose.
*This procedure could be performed on any dosing day followed by a minimum 30 minute washout before any aerosol dosing procedures. Alternatively the ^{81m}Kr ventilation scan could be carried out on a separate visit. (This procedure only occurred once).

### 9.5.2 Appropriateness of Measurements

All measurements performed in this study were standard measurements.

### 9.5.3 Primary Efficacy Variable(s)

To compare the emitted dose, delivered lung dose, and the central-to-peripheral (sC/P) lung deposition of radiolabeled treprostinil sodium delivered via the AERx Essence System versus the Nebu-Tec Optineb nebulizer using gamma scintigraphy.

In addition the following secondary efficacy variables were determined, the dose (μg) of treprostinil deposited in the lung, the percent dose (emitted and loaded) of radiolabeled treprostinil sodium in the oropharyngeal region from both devices, the percent loaded dose remaining in both devices and associated equipment e.g. mouthpiece.

### 9.5.4 Drug Concentration Measurements

To evaluate treprostinil plasma pharmacokinetics, 16 venous blood samples were drawn into 7.5 ml Potassium EDTA monovette tubes following each study dose (i.e., Essence and Optineb). Sampling occurred approximately 1 hour prior to dosing and at +2, +3, +5, +7, +10, +15, +20, +30, +60, +90, +120, +180, +240, +300, and +360 minutes after the start of each study dose. Thus, a total of 32 blood samples (˜250 mL) were collected for pharmacokinetic assessment over the two dosing days. Immediately upon sampling the sample was identified with a bar coded label bearing details of study number, subject number, sampling time point, sample type and a unique 9 digit identification number. The sample was separated by centrifugation at 1500×g and 4° C. for 10 minutes. Two equal aliquots of plasma/serum were transferred to 2 polypropylene tubes labelled identically to the original blood sample and stored at approximately −20° C. pending analysis. The time at which samples were taken, received into the separating room and placed in the freezer was recorded in the study documentation.

### 9.6 Data Quality Assurance

At the time the study was initiated, a representative of the Sponsor thoroughly reviewed the Final Protocol and CRFs with the Investigator and staff. During the course of the study the Monitor visited the centre regularly, to check the completeness of subject records, the accuracy of entries into the CRFs, the adherence to the Final Protocol and to ICH Good Clinical Practice, the progress of enrolment and also to ensure that study medication was being stored, dispensed and accounted for according to specifications. The Investigator and key study personnel were available to assist the Monitor during these visits.

The Investigator gave the Monitor access to relevant clinical records, to confirm their consistency with the CRF entries. No information in these records about the identity of the subjects left the study centre. The Sponsor maintained confidentiality of all subject records.

The study data was subject to an independent audit by the Quality Assurance Unit of Simbec Research Limited.

### 9.7 Statistical Methods Planned in the Protocol and Determination of Sample Size

### 9.7.1 Statistical and Analytical Plans

Simbec carried out the statistical analysis. Full details of the statistical analyses of the data were documented in an agreed statistical analysis plan, which was finalised prior to locking the database and subsequent analysis of the study data.

The randomization, sample size calculations, and statistical analyses for this study were conducted by Simbec Research Ltd. The primary analyses were based upon data from subjects who completed all study treatments and assessments according to the protocol. Secondary analysis used the “intent-to-treat” population that included subjects who had received at least one dose of study drug.

The primary analyses were to compare the dose-to-lung equivalence between the AERx Essence System versus the Nebu-Tec Optineb nebulizer. The secondary analysis assessed the central to peripheral ratio of deposition in the lungs, and to compare the total oropharyngeal deposition of drug between the AERx Essence System and the Nebu-Tec Optineb nebulizer.

### 10. Study Subjects

### 10.1 Disposition of Subjects

Twenty-two (22) volunteers were screened for the study. Fourteen (14) subjects received study medication. A total of fourteen (14) subjects completed the study successfully as per protocol.

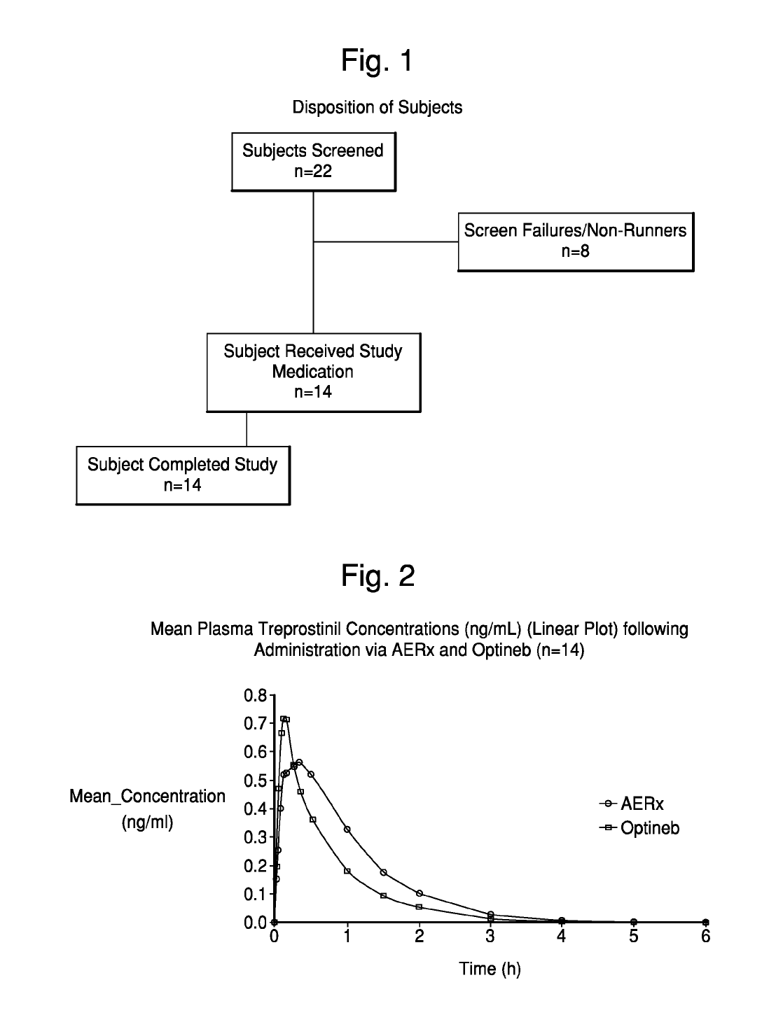

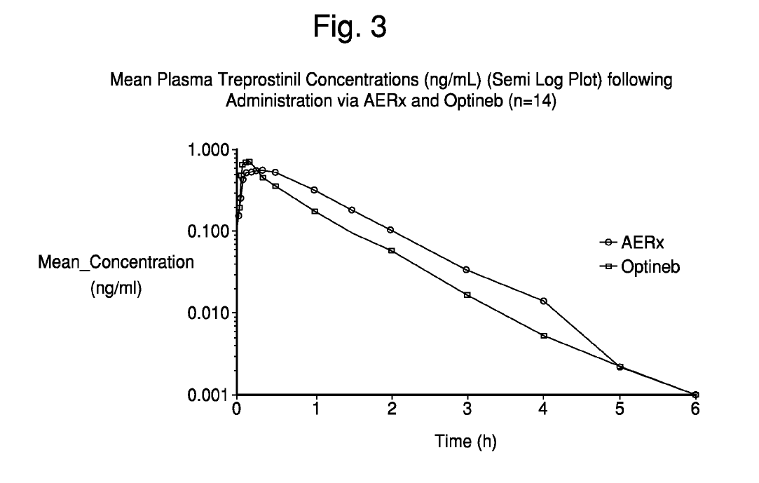

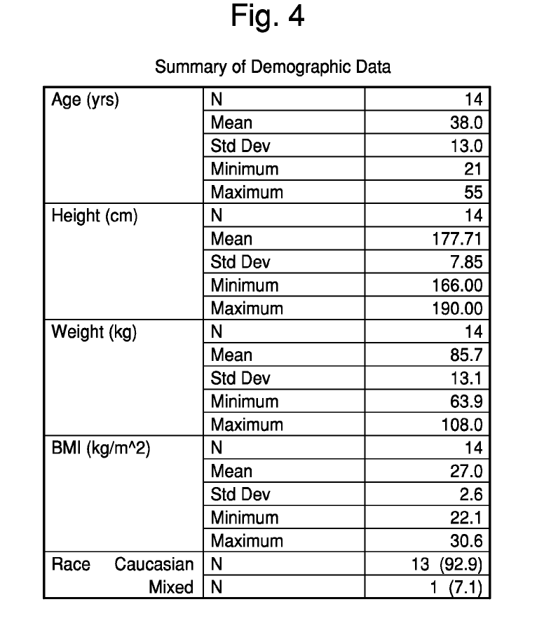

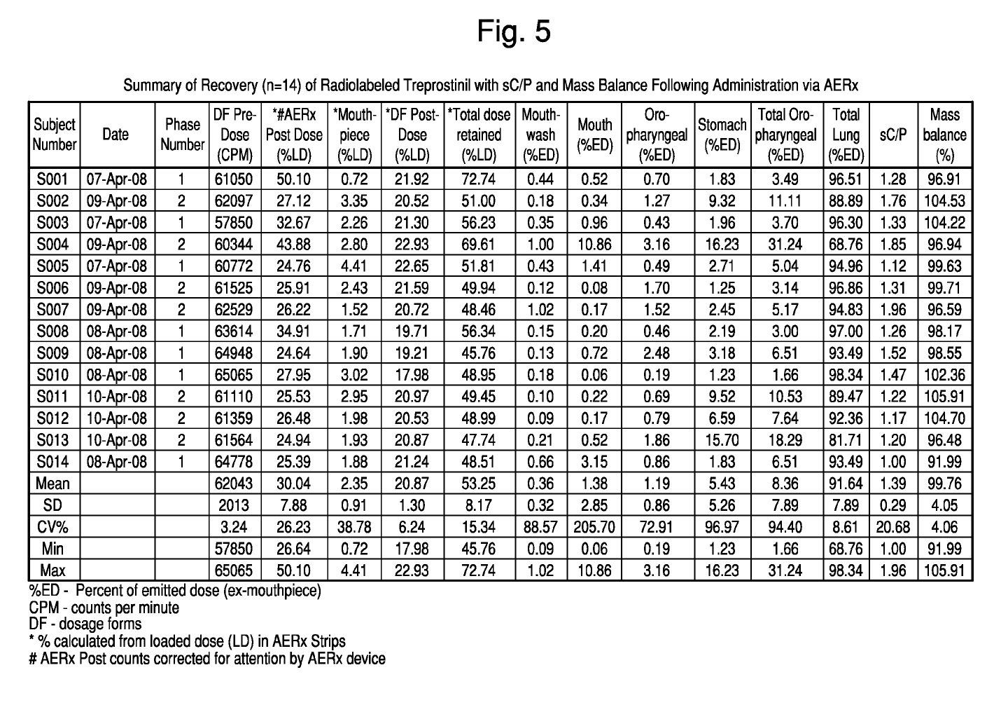

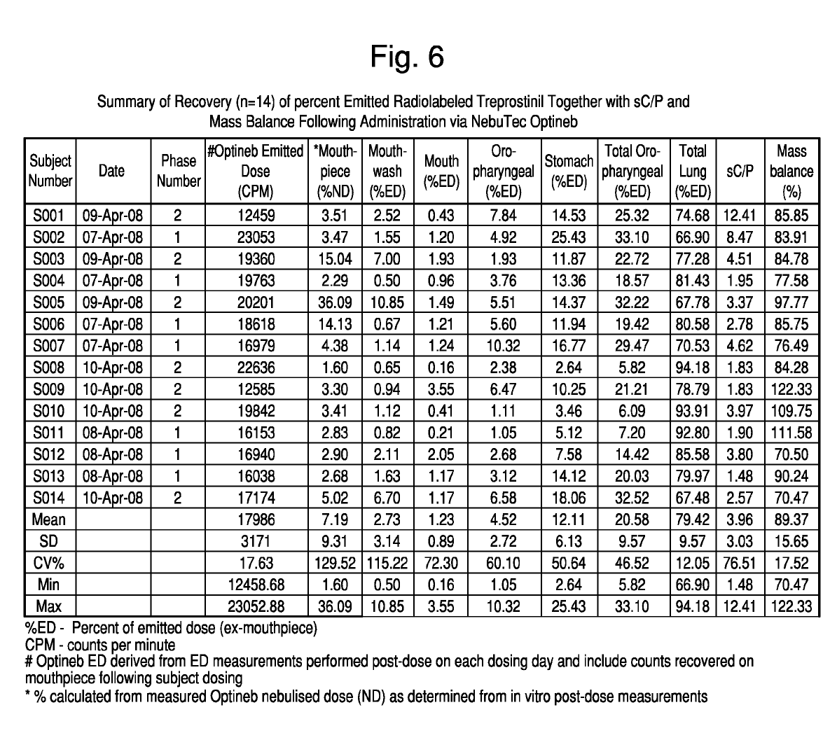

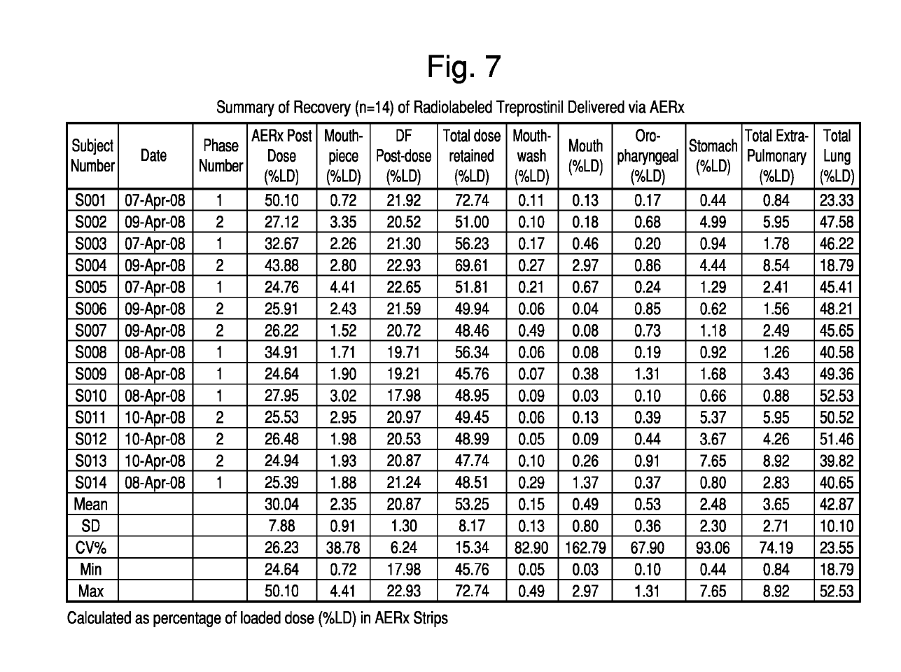

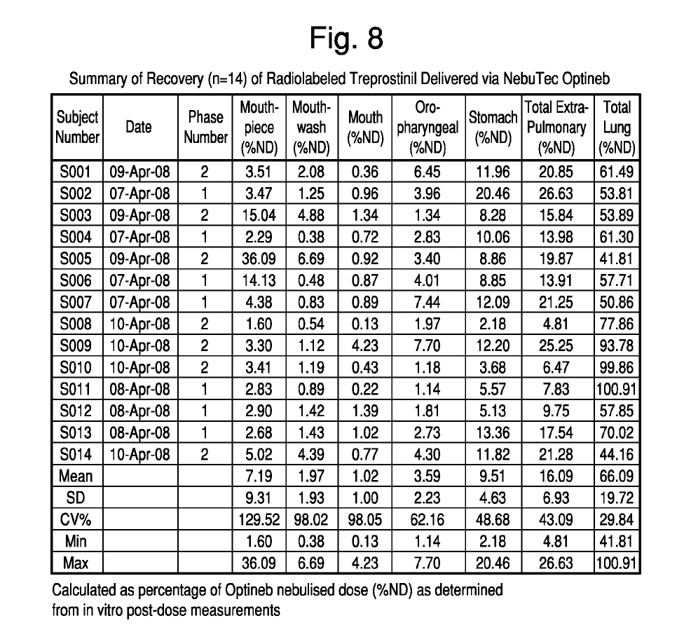

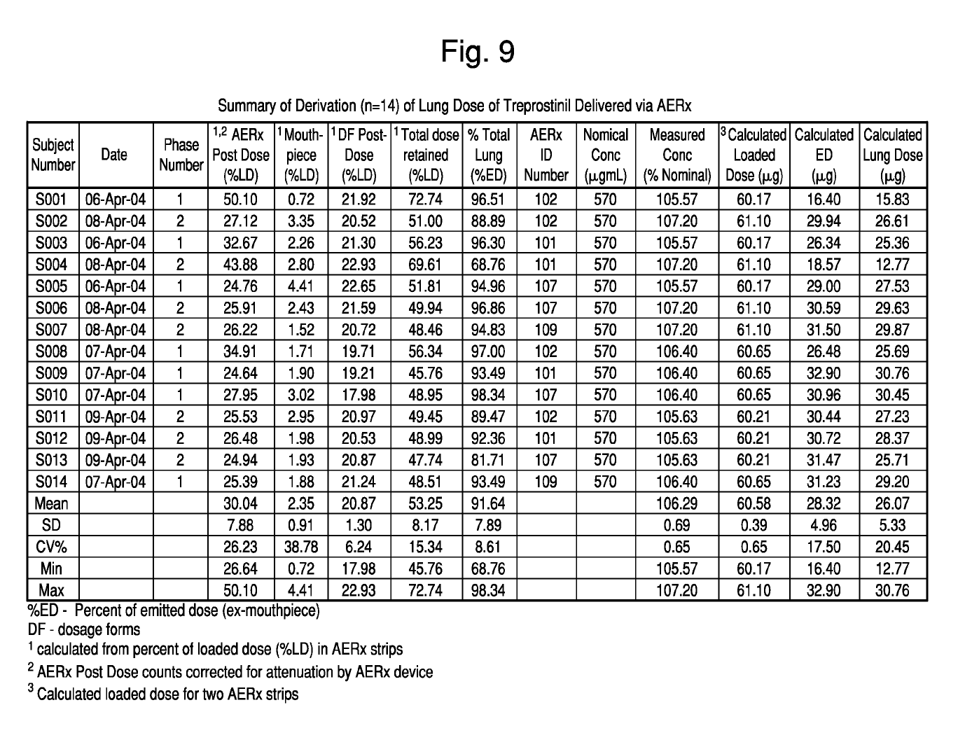

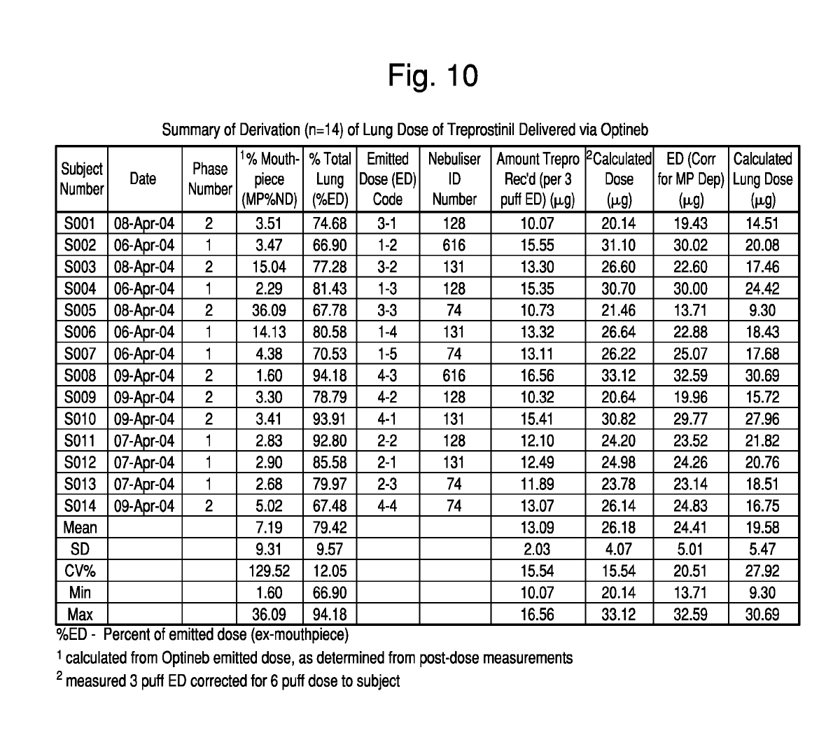

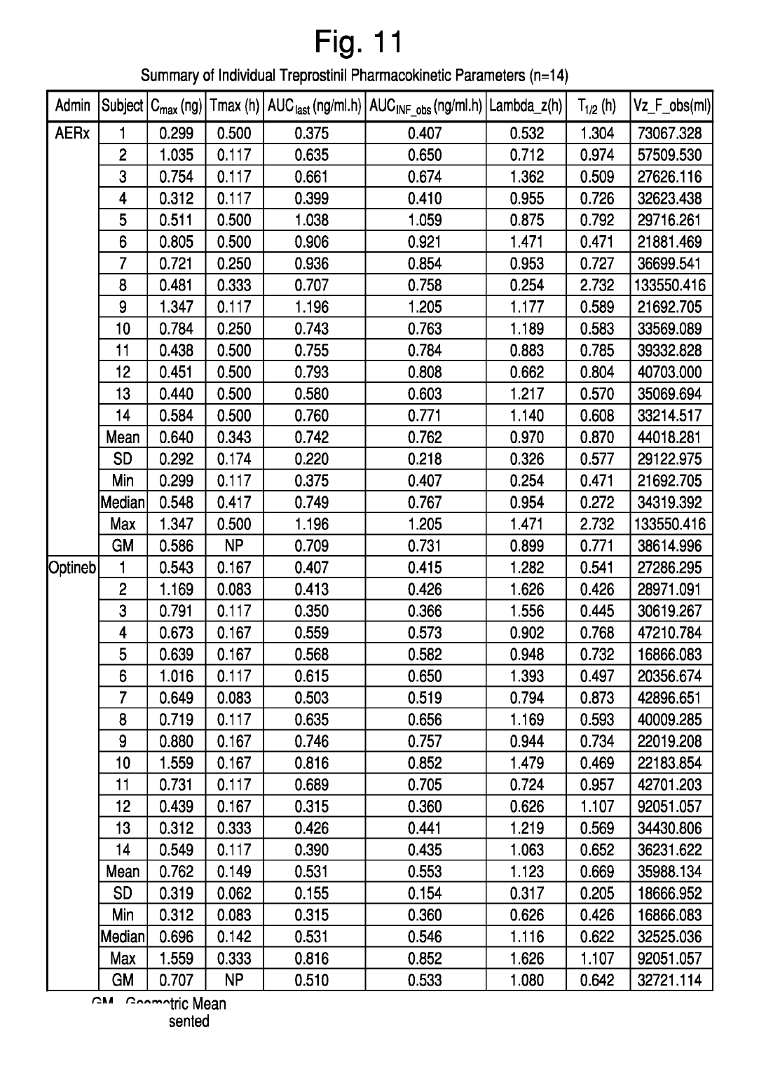

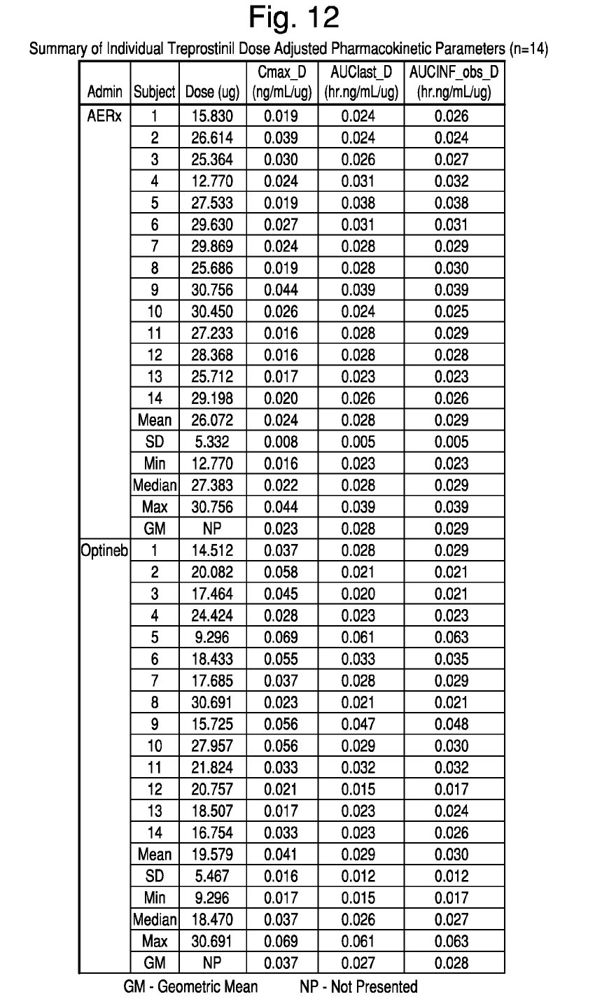

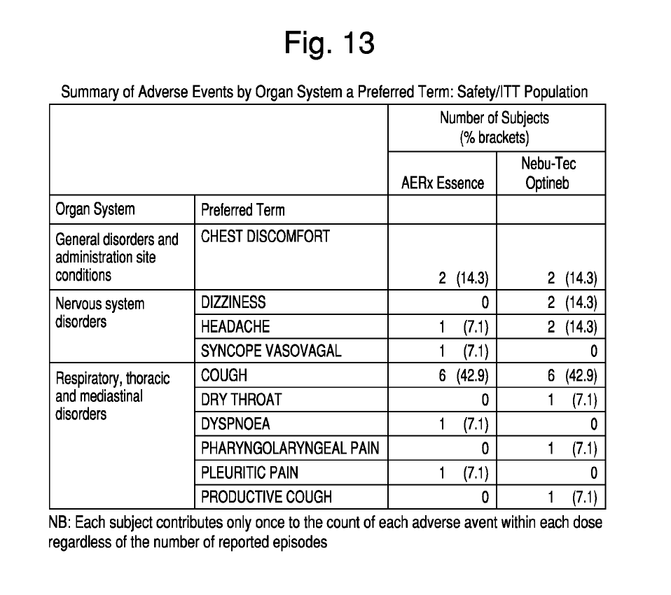

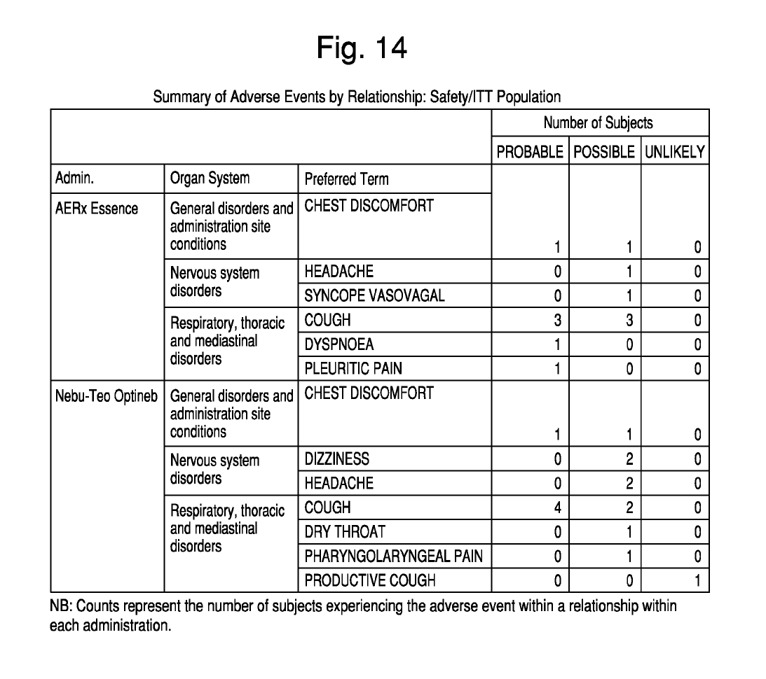

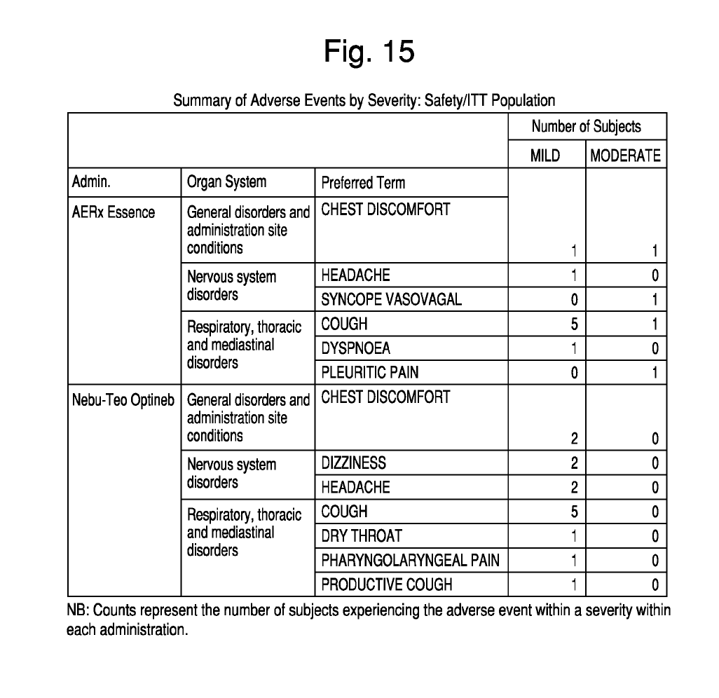

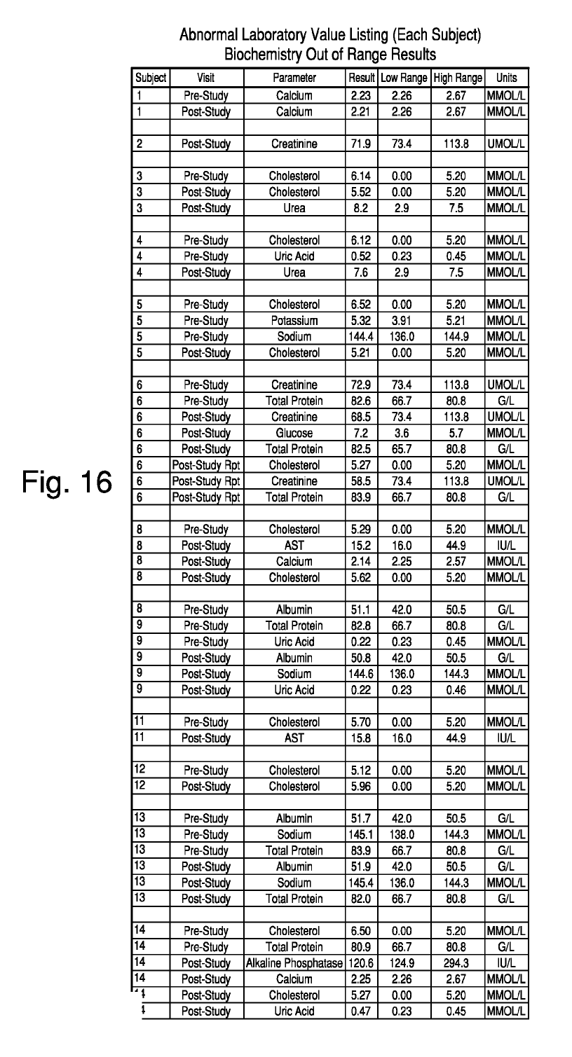

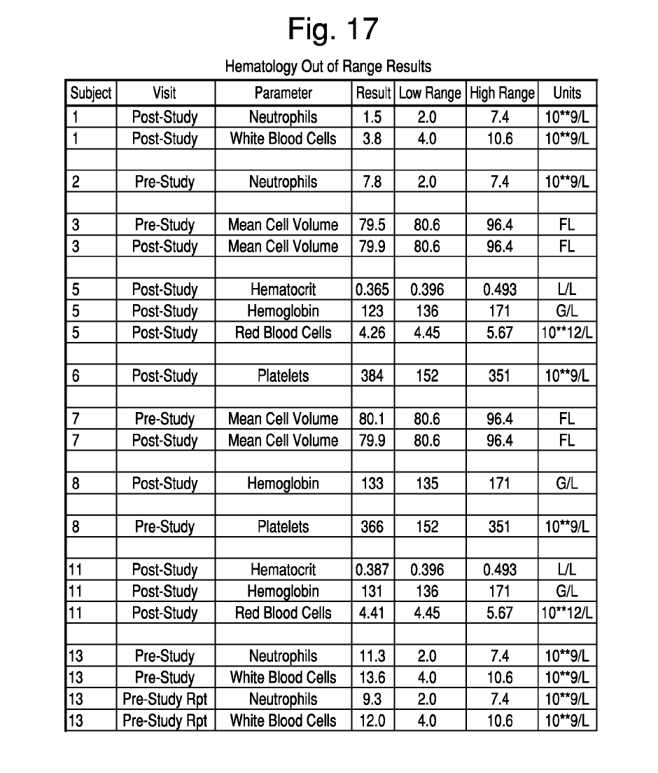

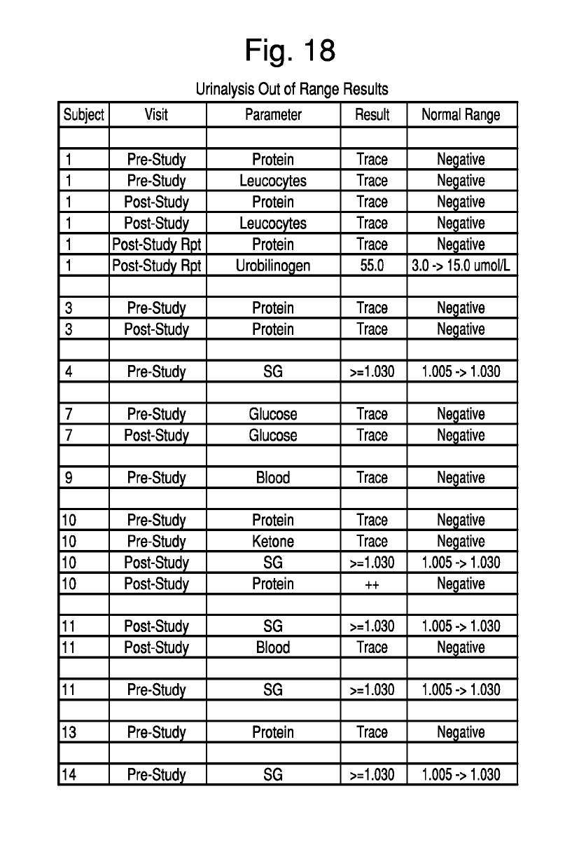

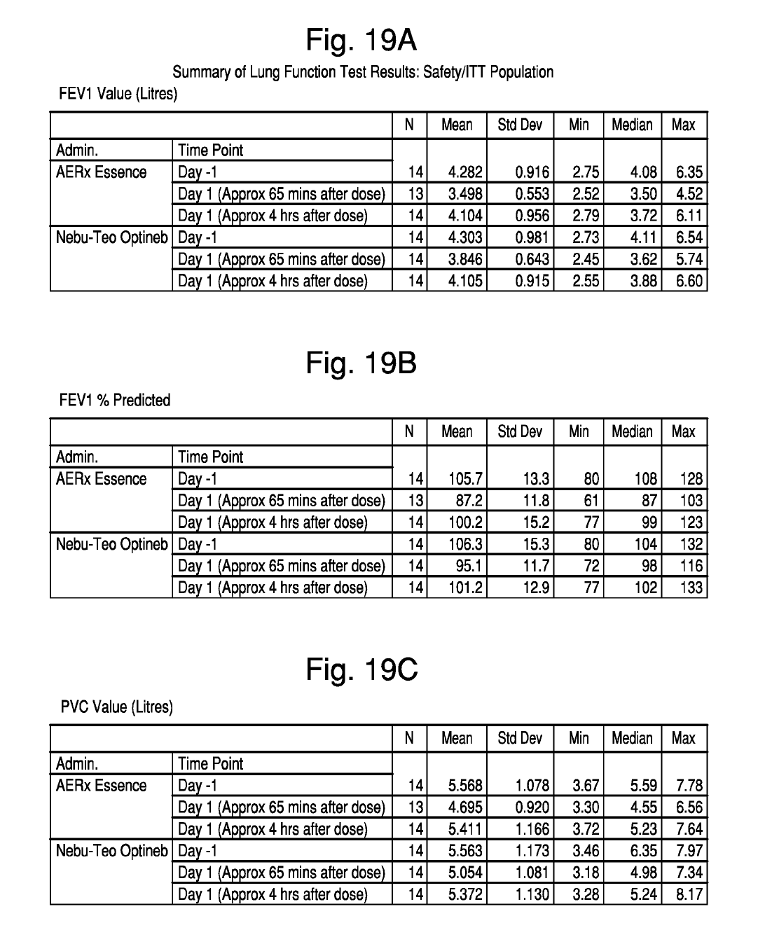

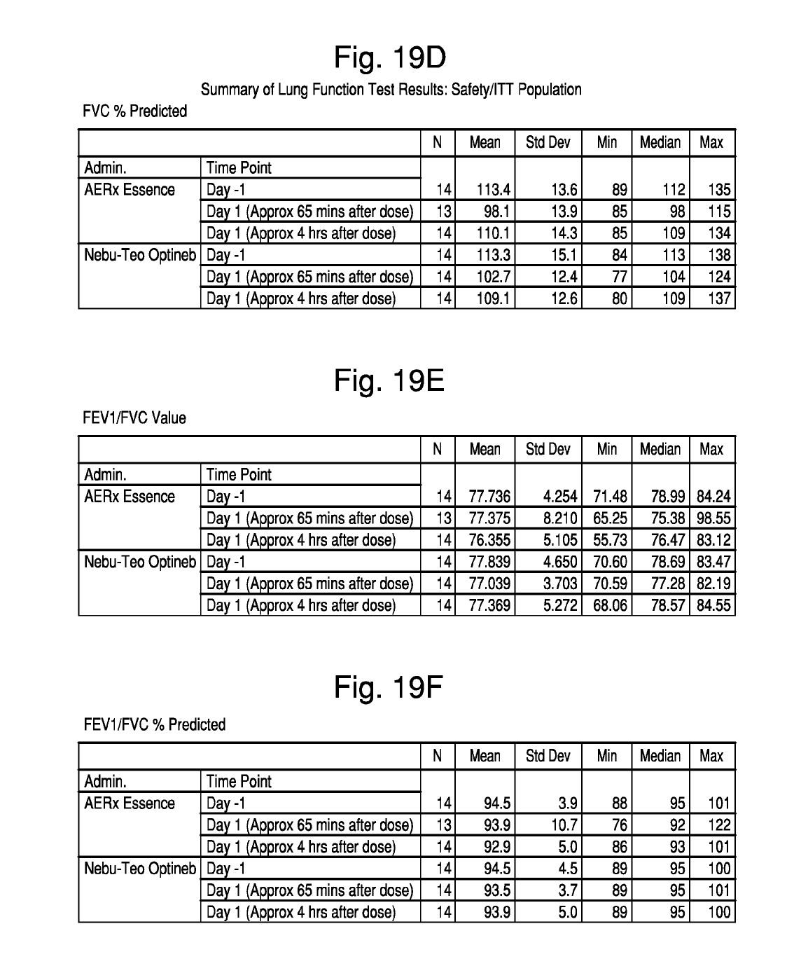

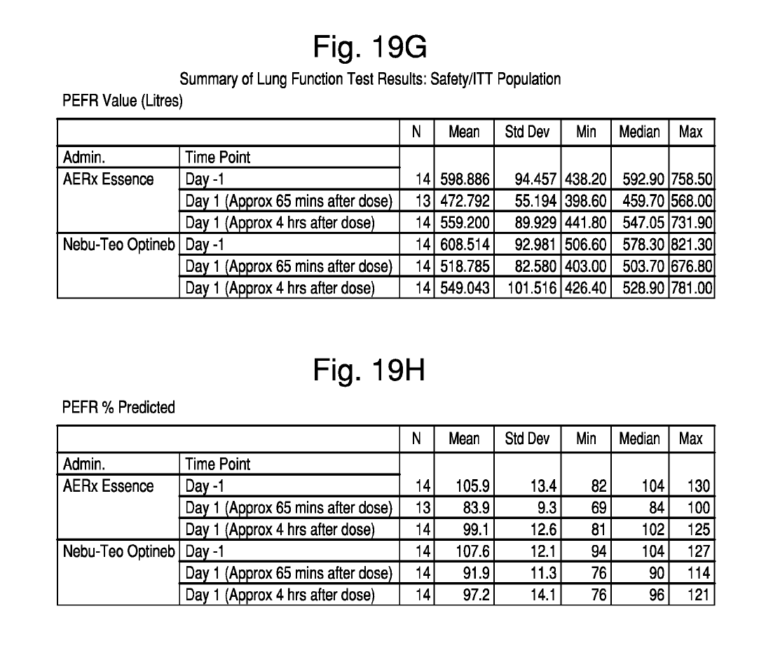

A summary of the disposition of all subjects is provided in FIG. 1.

### 10.2 Protocol Deviations

A number of file notes were recorded. These are summarised below:

The study protocol indicated that 16 volunteers should be randomised to the study. During the clinical phase of the study only 14 subjects were randomised, due to volunteer recruitment issues. A decision was made by the sponsor that 14 randomised volunteers would be adequate for the analysis. The samples size stated in the protocol was not statistically powered and therefore the integrity of the study was not affected (Ref: 10APR08/AJ/02).

Repeat blood pressures were conducted on Day −1, and noted on the additional notes page within the CRF. One of the exclusion criteria for the study is ‘History of orthostatic hypotension’. Unless this was documented in the Volunteers Master File (VMF) it was considered to be unlikely that the volunteer would give this information to a research physician when questioned. It was decided that on arrival at Simbec a standing blood pressure would be conducted as well as a supine blood pressure to ensure that there is no evidence of orthostatic hypotension.

### 11. Efficacy/Pharmacokinetic/Pharmacodynamic Evaluation

### 11.1 Data Sets Analysed

All fourteen (14) subjects who were eligible at screening and randomised on the first dosing day, received one dose of the study drug were therefore included in the Safety Population.

All fourteen (14) subjects completed the two study periods and had sufficient blood samples taken to obtain a plasma concentration by time profile and were therefore included in the Pharmacokinetic population and Gamma Scintigraphy population.

### 11.2 Demographic and Other Baseline Characteristics

At pre-study the subjects had a mean age of 38.0 years (SD 13.0), a mean weight of 85.7 kg (SD 13.1) and a mean height of 177.71 centimetres (SD 7.85).

### 11.3 Measurements of Treatment Compliance

All patches were administered and removed by a Research Physician and checked by a second member of staff. The patches were checked regularly over the 72 hour period by clinical staff, ensuring that subjects were compliant with treatment.

### 11.4 Efficacy & Pharmacokinetic Results and Tabulations of Individual Subject Data

### 11.4.1 Analysis of Efficacy (Radiolabel Distribution) & Pharmacokinetics

### 11.4.1.1 Efficacy (Radiolabel Distribution)

TABLE 11.4.1.1.1
Summary of Recovery (n = 14) of Percent Emitted Radiolabeled Treprostinil, Together
with sC/P and Mass Balance Following Administration via AERx and Nebu-Tec Optineb.

| | | Mouth- | | Oro- | | Total Oro- | Total | | Mass |
|---|---|---|---|---|---|---|---|---|---|
| Admin | | wash | Mouth | pharyngeal | Stomach | pharyngeal | Lung | sC/P | balance |
| AERx | Mean | 0.36 | 1.38 | 1.19 | 5.43 | 8.36 | 91.64 | 1.39 | 99.76 |
| | D | 0.32 | 2.85 | 0.86 | 5.26 | 7.89 | 7.89 | 0.29 | 4.05 |
| | CV(%) | 88.57 | 205.70 | 72.91 | 96.97 | 94.40 | 8.61 | 20.68 | 4.06 |
| | Min | 0.09 | 0.06 | 0.19 | 1.23 | 1.66 | 68.76 | 1.00 | 91.99 |
| | Max | 1.02 | 10.86 | 3.16 | 16.23 | 31.24 | 98.34 | 1.96 | 105.91 |
| Optineb | Mean | 2.73 | 1.23 | 4.52 | 12.11 | 20.58 | 79.42 | 3.96 | 89.37 |
| | SD | 3.14 | 0.89 | 2.72 | 6.13 | 9.57 | 9.57 | 3.03 | 15.65 |
| | CV(%) | 115.22 | 72.30 | 60.10 | 50.64 | 46.52 | 12.05 | 76.51 | 17.52 |
| | Min | 0.50 | 0.16 | 1.05 | 2.64 | 5.82 | 66.90 | 1.48 | 70.47 |
| | Max | 10.85 | 3.55 | 10.32 | 25.43 | 33.10 | 94.18 | 12.41 | 122.33 |

The mean recovery of deposited radioactivity as percentage of the emitted dose (% ED) i.e. radiolabeled aerosol exiting the AERx or Optineb mouthpiece is shown in Table 11.4.1.1.1. The individual data are shown in Section 14.2, Tables 14.2.1.1 and 14.2.1.2. For both devices the majority of the emitted aerosol was deposited in the lungs the mean value for AERx was 91.64% (±7.89%) and 79.42% (±9.57%) for Optineb. Analysis of variance (ANOVA) was performed and the difference between the least squares (LS) means (95% confidence interval (CI)), was 12.22% (5.29-19.15%) (Table 11.4.1.1.4) indicating that the fraction deposited in the lung following AERx administration was statistically significantly greater than that following Optineb. The coefficient of variation (CV) associated with AERx lung deposition was 8.61% compared to 12.05% for Optineb.

The mean total oropharyngeal deposition i.e. sum of mouthwash, mouth, oropharynx and stomach, for AERx was 8.36% (±7.89%) and 20.58 5 (±9.57%) for Optineb. The difference in the LS means (Table 11.4.1.1.4) was −12.22 (−19.15-−5.29) which indicated that a statistically significantly greater fraction was deposited in the oropharyngeal region following Optineb dosing compared to AERx.

The retention of radioactivity on the mouthpiece of each device was expressed in terms of the % loaded dose (radioactivity). The mean value for AERx (Table 11.4.1.1.2) was 2.35% (±0.91%), and 7.19% (±9.31%) for Optineb (Table 11.4.1.1.3). The difference between the LS means (Table 11.4.1.1.4) was −4.84% (−9.92-0.23%), indicating that there was no statistically significant difference between the devices. The CV values however indicated that deposition in this location was more variable for Optineb (CV 129.52) compared to 38.78% for AERx.

The pattern of radiolabel distribution within the lung was described in terms of the central to peripheral ratio, normalised for Krypton-81m gas distribution (sC/P). The mean value for AERx was 1.39 (±0.29) and the mean sC/P for Optineb was 3.96 (±3.03) (Table 11.4.1.1.1). The difference between the LS mean values (Table 11.4.1.1.4) was −2.57 (−4.37-−0.78) indicating that the difference between the two devices was statistically significantly different, i.e. radiolabel distribution within the lung was more homogeneous following AERx compared to Optineb. The coefficient of variation associated with sC/P for AERx was 20.68%, in contrast, for Optineb the CV was 76.51% (Table 11.4.1.1.1).

The mass balance data reported in Table 11.4.1.1.1 showed that the tissue attenuation correction factors, derived from individual transmission images, were accurate. The mean mass balance value for total radioactivity recovered following AERx dosing was 99.76% (±4.05%) and 89.37% (±15.65%) following Optineb delivery.

TABLE 11.4.1.1.2
Summary of Distribution (n = 14) of Radiolabeled Treprostinil Delivered via AERx

| | | | | ¹Total | | | | | |
|---|---|---|---|---|---|---|---|---|---|
| | ^{1,2}AERx | ¹Mouth- | ¹DF | dose | % Total | Measured | ³Calculated | | Calculated |
| | Post Dose | piece | Post-dose | retained | Lung | Conc | Loaded | Calculated | Lung Dose |
| AERx | (% LD) | (% LD) | (% LD) | (% LD) | (% ED) | (% Nominal) | Dose (μg) | ED (μg) | (μg) |
| Mean | 30.04 | 2.35 | 20.87 | 53.25 | 91.64 | 106.29 | 60.58 | 28.32 | 26.07 |
| SD | 7.88 | 0.91 | 1.30 | 8.17 | 7.89 | 0.69 | 0.39 | 4.96 | 5.33 |
| CV(%) | 26.23 | 38.78 | 6.24 | 15.34 | 8.61 | 0.65 | 0.65 | 17.50 | 20.45 |
| Min | 24.64 | 0.72 | 17.98 | 45.76 | 68.76 | 105.57 | 60.17 | 16.40 | 12.77 |
| Max | 50.10 | 4.41 | 22.93 | 72.74 | 98.34 | 107.20 | 61.10 | 32.90 | 30.76 |

% ED—Percent of emitted dose (ex-mouthpiece)
DF—dosage forms
¹% calculated from percent of loaded dose (% LD) in AERx strips
²AERx Post Dose counts corrected for attenuation by AERx device
³Calculated loaded dose for two AERx strips

TABLE 11.4.1.1.3
Summary of Distribution (n = 14) of Radiolabeled Treprostinil Delivered via Optineb..

| | ¹Mouth-piece | Total Lung | Amount Trepro Rec'd | ²Calculated | ED (Corr for | Calculated Lung |
|---|---|---|---|---|---|---|
| Optineb | (MP) (% ND) | (% ED) | (per 3 puff ED) (μg) | Dose (μg) | MP Dep) (μg) | Dose (μg) |
| Mean | 7.19 | 79.42 | 13.09 | 26.18 | 24.41 | 19.58 |
| SD | 9.31 | 9.57 | 2.03 | 4.07 | 5.01 | 5.47 |
| CV(%) | 129.52 | 12.05 | 15.54 | 15.54 | 20.51 | 27.92 |
| Min | 1.60 | 66.90 | 10.07 | 20.14 | 13.71 | 9.30 |
| Max | 36.09 | 94.18 | 16.56 | 33.12 | 32.59 | 30.69 |

% ED—Percent of emitted dose (ex-mouthpiece)
¹% calculated from Optineb nebulised dose (ND), as determined from in vitro post-dose measurements
²measured 3 puff ED corrected for 6 puff dose to subject

The dose to lung, in terms of μg of treprostinil was calculated following adjustment of the fraction delivered to the lung for retention of dose within each device and for the measured concentration of the dosing solutions. The mean calculated dose to lung for AERx was 26.07 μg (±5.33 μg) of treprostinil (Table 11.4.1.1.2), the mean dose following Optineb administration was 19.58 μg (±5.47 μg) (Table 11.4.1.1.3).

TABLE 11.4.1.1.4
Statistical Analysis of Treprostinil Deposition Data (n = 14)

| | AERx | Nebu-Tec | AERx − Nebu-Tec |
|---|---|---|---|
| | LSmeans | | Difference (95% C.I.) |
| % emitted dose in lung | 91.64 | 79.42 | 12.22 (5.29-19.15) |
| % emitted dose in | 8.36 | 20.58 | −12.22 (−19.15-−5.29) |
| oropharyngeal region | | | |
| % loaded dose in | 2.35 | 7.19 | −4.84 (−9.92-0.23) |
| mouthpiece | | | |
| Penetration Index (sC/P) | 1.39 | 3.96 | −2.57 (−4.37-−0.78) |

Output File: stats_depos, produced: 12JUN2008 15:32, Final

TABLE 11.4.1.2.1
Summary of Pharmacokinetic Parameters following Administration
of treprostinil via AERx and NebuTec Optineb (n = 14).

| | | Cmax | Tmax | AUCₗₐₛₜ | AUC_{INF}_{_{—}}_{obs} | Lambda_z | T_{1/2} | Vz_F_obs |
|---|---|---|---|---|---|---|---|---|
| Admin | Subject | (ng/mL) | (h) | (ng/ml · h) | (ng/ml · h) | (h) | (h) | (ml) |
| AERx | Mean | 0.640 | 0.343 | 0.742 | 0.762 | 0.970 | 0.870 | 44018.281 |
| | SD | 0.292 | 0.174 | 0.220 | 0.218 | 0.326 | 0.577 | 29122.975 |
| | Min | 0.299 | 0.117 | 0.375 | 0.407 | 0.254 | 0.471 | 21692.705 |
| | Median | 0.548 | 0.417 | 0.749 | 0.767 | 0.954 | 0.727 | 34319.392 |
| | Max | 1.347 | 0.500 | 1.196 | 1.205 | 1.471 | 2.732 | 133550.416 |
| | GM | 0.586 | N/P | 0.709 | 0.731 | 0.899 | 0.771 | 38614.996 |
| Optineb | Mean | 0.762 | 0.149 | 0.531 | 0.553 | 1.123 | 0.669 | 35988.134 |
| | SD | 0.319 | 0.062 | 0.155 | 0.154 | 0.317 | 0.205 | 18666.952 |
| | Min | 0.312 | 0.083 | 0.315 | 0.360 | 0.626 | 0.426 | 16866.083 |
| | Median | 0.696 | 0.142 | 0.531 | 0.546 | 1.116 | 0.622 | 32525.036 |
| | Max | 1.559 | 0.333 | 0.816 | 0.852 | 1.626 | 1.107 | 92051.057 |
| | GM | 0.707 | N/P | 0.510 | 0.533 | 1.080 | 0.642 | 32721.114 |

GM—geometric mean
N/P—not presented

The derived pharmacokinetic parameters are shown in Table 11.4.1.2.1. The mean Cₘₐₓ (ng/mL) for the AERx treatment was 0.640 ng/mL (±0.292 ng/mL) and for Optineb the corresponding value was 0.762 (±0.319 ng/mL). The ratio of the geometric LS means (90% CI) was 82.88 (68.99-99.56) (Table 11.4.1.2.2) indicating that there was a statistically significant difference between the Cₘₐₓ values for the two treatments.

The mean Tₘₐₓ values (h) were 0.343 h (±0.174 h) and 0.149 (±0.062 h) for AERx and Optineb respectively (Table 11.4.1.2.1). The difference in the median values (95% CI) for Tₘₐₓ (Table 11.4.1.2.2) was 11.5 min (5.0-20.0). The p value was 0.0046 indicating a statistically significant difference between the AERx and Optineb administrations.

The mean AUC_{T} values (ng.h/mL, ±SD) for AERx and Optineb were 0.742 ng.h/mL (0.220 ng.h/mL) and 0.531 ng.h/mL (0.155 ng.h/mL) respectively. The mean AUC_{I} values (ng.h/mL, ±SD) for AERx and Optineb were 0.762 ng.h/mL (0.218 ng.h/mL) and 0.553 ng.h/mL (0.154 ng.h/mL) respectively (Table 11.4.1.2.1).

The ratio of the geometric LS means (90% CI) for AUC_{T} was 139.11 (116.90-165.54) showing that this AUC parameter was statistically significantly greater following AERx dosing than that following Optineb administration. A similar finding was observed for AUC_{I}, the ratio of the geometric LS means was 137.15 (117.02-160.75) (Table 11.4.1.2.2).

The mean (±SD) elimination rate constant (h) for treprostinil following AERx dosing was 0.970 h (±0.326 h) and 1.123 h (±0.317 h) for Optineb. The mean (±SD) elimination half life (h) for treprostinil was 0.870 h (±0.577 h) and 0.669 h (±0.205 h) for AERx and Optineb respectively (Table 11.4.1.2.1).

The volume of distribution (Vd) for treprostinil is shown in Table 11.4.1.2.1. The mean Vd (mL±SD) was 44018.281 mL (±29122.975 mL) following AERx and 35988.134 mL (±18666.952 mL) following Optineb dosing (Table 11.4.1.2.1).

TABLE 11.4.1.2.2
Statistical Analysis (n = 14) of Treprostinil Pharmacokinetic Parameters

| | | | AERx/ | |
|---|---|---|---|---|
| | AERx | Nebu-Tec | Nebu-Tec | |
| | Geometric LS means | | Ratio (%) | 90% C.I. |
| Cmax (ng/ml) | 0.59 | 0.71 | 82.88 | 68.99-99.56 |
| AUC_{T} (ng · h/ml) | 0.71 | 0.51 | 139.11 | 116.90-165.54 |
| AUC_{I} (ng · h/ml) | 0.73 | 0.53 | 137.15 | 117.02-160.75 |
| | | | Median Diff. | |
| | Median | | (p-value*) | 95% C.I. |
| Tmax (min.) | 25.0 | 8.5 | 11.5 | 5.0-20.0 |
| | | | (0.0046) | |

Output File: stats, produced: 12JUN2008 12:51, Final
*Wilcoxon Matched Pairs Test

TABLE 11.4.1.2.3
Summary of Dose Normalised Pharmacokinetic Parameters
following Administration of treprostinil via AERx and
NebuTec Optineb (n = 14).

| | | | Cmax_D | AUClast_D | |
|---|---|---|---|---|---|
| | | Dose | (ng/mL/ | (hr · ng/ | AUCINF_obs_D |
| Admin | Subject | (ug) | ug) | ml/ug) | (hr · ng/ml/ug) |
| AERx | Mean | 26.072 | 0.024 | 0.028 | 0.029 |
| | SD | 5.332 | 0.008 | 0.005 | 0.005 |
| | Min | 12.770 | 0.016 | 0.023 | 0.023 |
| | Median | 27.383 | 0.022 | 0.028 | 0.029 |
| | Max | 30.756 | 0.044 | 0.039 | 0.039 |
| | GM | NP | 0.023 | 0.028 | 0.029 |
| Optineb | Mean | 19.579 | 0.041 | 0.029 | 0.030 |
| | SD | 5.467 | 0.016 | 0.012 | 0.012 |
| | Min | 9.296 | 0.017 | 0.015 | 0.017 |
| | Median | 18.470 | 0.037 | 0.026 | 0.027 |
| | Max | 30.691 | 0.069 | 0.061 | 0.063 |
| | GM | NP | 0.037 | 0.027 | 0.028 |

GM—geometric mean
N/P—not presented

The pharmacokinetic parameters Cₘₐₓ, AUC_{T} and AUC_{I} were normalised for the dose delivered to the lung, as determined from the scintigraphy data (Tables 14.2.1.5 & 14.2.1.6).

The mean dose normalised Cₘₐₓ (ng/mL/μg) values were 0.024 (±0.08) and 0.041 (±0.016) for AERx and Optineb respectively (Table 11.4.1.2.3). The ratio of the geometric LS means (Table 11.4.1.2.4) for dose normalised Cₘₐₓ was 61.51 (52.53-72.02) indicating that this value was statistically significantly less following AERx administration than Optineb.

The mean dose normalised AUC_{T} (hr.ng/mL/μg) values (±SD) for AERx and Optineb were 0.028 (0.005) and 0.029 (0.012) respectively (Table 11.4.1.2.3). The mean dose normalised AUC_{I} (hr.ng/mL/μg) values (±SD) for AERx and Optineb were 0.029 (0.005) and 0.030 (0.012) respectively (Table 11.4.1.2.3).

The ratio of the geometric LS means (90% CI) for dose normalised AUC_{T} (Table 11.4.1.2.4) was 103.24 (90.63-117.61) indicating that following normalisation for the dose delivered to the lung there was no statistically significant difference between the treatments. A similar observation was made for AUC_{I}, the ratio of the geometric LS means (90% CI) was 101.79 (90.04-115.07) i.e. no statistically significant difference between the values for the two treatments.

TABLE 11.4.1.2.4
Statistical Analysis (n = 14) of Treprostinil Dose-Normalised
Pharmacokinetic Parameters

| | AERx | Nebu-Tec | | |
|---|---|---|---|---|
| | Geometric | | AERx/Nebu-Tec | |
| | LS means | | Ratio (%) | 90% C.I. |
| Cₘₐₓ (ng/ml) | 0.023 | 0.037 | 61.51 | 52.53-72.02 |
| AUC_{T} (ng · h/ml) | 0.028 | 0.027 | 103.24 | 90.63-117.61 |
| AUC_{I} (ng · h/ml) | 0.029 | 0.028 | 101.79 | 90.04-115.07 |

Output File: stats, produced: 12JUN2008 12:51, Final
NB: Pharmacokinetic parameters dose-normalised for the calculated dose to lung (μg).

Conclusions

### 11.4.7.1 Efficacy (Radiolabel Distribution)

For both devices the majority of the emitted aerosol was deposited in the lungs, however the mean value for AERx (91.64%±7.89%) was statistically significantly greater than that for Optineb (79.42%±9.57%). The coefficient of variation (CV) associated with AERx lung deposition was 8.61% compared to 12.05% for Optineb indicating less variability in the dose delivered to the lung.

The mean total oropharyngeal deposition was statistically significantly less following AERx (8.36%, ±7.89%) compared to Optineb (20.58 5%, ±9.57%).

There was no statistically significant difference in the retention of radioactivity on the mouthpiece of each device (% loaded dose). The mean values were 2.35% (±0.91%), and 7.19% (±9.31%) for AERx and Optineb respectively.

The pattern of radiolabel distribution within the lungs (sC/P) was found to be more homogeneous for AERx (1.39, ±0.29) than for Optineb (3.96, ±3.03) which was deposited to a greater extent in the central airways. The difference between the two treatments was statistically significant.

Mass balance data showed that the tissue attenuation correction factors, derived from individual transmission images, were accurate. The mean mass balance value for total radioactivity recovered following AERx dosing was 99.76% (±4.05%) and 89.37% (±15.85%) following Optineb delivery.

### 11.4.7.2 Pharmacokinetics

The mean Cₘₐₓ (ng/mL) following AERx dosing (0.640 ng/mL, ±0.292 ng/mL) was statistically significantly less than that following Optineb administration (0.762 ng/mL, ±0.319 ng/mL).

The time to Cₘₐₓ i.e. Tₘₐₓ for the two treatments was also statistically significantly different, the mean value for AERx (0.343 h, ±0.174 h) was significantly longer than that of Optineb (0.149 h, ±0.062 h).

The mean AUC_{T} and AUC_{I} values (ng.h/mL) for AERx were statistically significantly smaller than those calculated for Optineb. The mean AUC_{T} (ng/mL.h) values were 0.742 ng/mL.h (±0.220 ng/mL.h)) and 0.531 ng/mL.h (±0.155 ng/mL.h) for AERx and Optineb respectively. The mean AUC_{I} values (ng.h/mL) were 0.762 ng/mL.h (±0.218 ng/mL.h)) and 0.553 ng/mL.h (±0.154 ng/mL.h) for AERx and Optineb respectively.

### 11.4.7.3 Integrated Efficacy (Radiolabel Distribution) and Pharmacokinetics Conclusions

It may be concluded that the difference in drug deposition patterns within the lung influenced the absorption of the treprostinil.

The mean Tₘₐₓ for the more homogeneous AERx deposition (0.343 h) was significantly longer than that for the more centrally deposited Optineb deposition (0.149 h).

The ratio of the dose adjusted Cₘₐₓ (geometric LS means) was 61.51%, the non-dose adjusted ratio was 82.88%. Thus, despite a greater dose to lung via AERx the subsequent peak plasma concentration was lower than that observed following Optineb dosing.

Statistical analysis of dose adjusted AUC parameters i.e. AUC_{T} and AUC_{I} showed there were no statistically significant differences between the treatments, in contrast to the findings for the non-dose adjusted parameters. This finding indicates that despite a smaller dose to lung following Optineb administration the extent of drug absorption from the more central deposition exceeded that of the more peripheral distribution following AERx deposition.

### 12. Safety Evaluation

### 12.1 Extent of Exposure

A total of fourteen (14) subjects were exposed to treprostinil sodium on two occasions.

### 12.2 Adverse Events (AES)

### 12.2.1 Brief Summary of Adverse Events

There were no adverse events reported prior to dosing with the study medication. There were no Serious Adverse Events (SAE's) or Suspected Unexpected Serious Adverse Reactions (SUSAR's) reported during the study.

During the study there were a total of 27 treatment emergent adverse events were reported by 9 subjects. Fifteen (15) adverse events were recorded following administration of treprostinil sodium via the AERx Essence System. Twelve (12) adverse events were recorded following administration of treprostinil sodium via the Nebu-Tec Optineb.

### 12.2.2 Display of Adverse Events

Summary of adverse events by organ system and preferred term including number of subjects who experienced adverse events by organ system is provided in Table 12.2-1. The Summary of Adverse Events by Relationship is provided in Table 12.2-2.

TABLE 12.2-1
Summary of Adverse Events by Organ System & Preferred Term:
Safety/ITT Population

| | | Number of Subjects | |
|---|---|---|---|
| | | (% brackets) | |
| | | AERx | Nebu-Tec |
| Organ System | Preferred Term | Essence | Optineb |
| General Disorders | CHEST DISCOMFORT | 2 (14.3) | 2 (14.3) |
| and administration | | | |
| site conditions | | | |
| Nervous System | DIZZINESS | 0 | 2 (14.3) |
| Disorders | HEADACHE | 1 (7.1) | 2 (14.3) |
| | SYNCOPE VASOVAGAL | 1 (7.1) | 0 |
| Respiratory, | COUGH | 6 (42.9) | 6 (42.9) |
| thoracic and | DRY THROAT | 0 | 1 (7.1) |
| mediastinal | DYSPNOEA | 1 (7.1) | 0 |
| disorders | PHARYNGOLARYNGEAL | 0 | 1 (7.1) |
| | PAIN | | |
| | PLEURITIC PAIN | 1 (7.1) | 0 |
| | PRODUCTIVE COUGH | 0 | 1 (7.1) |

NB: Each subject contributes only once to the count of each adverse event within each dose regardless of the number of reported episodes
Output File: tab_ae_prf; Produced: 07JUN2008 14:23; Final

12.2-2 Summary of Adverse Events by Relationship: Safety/ITT Population

| | | | Number of Subjects | | |
|---|---|---|---|---|---|
| Admin. | Organ Systems | Preferred Type | PROBABLE | POSSIBLE | UNLIKELY |
| AERx Essence | General | CHEST DISCOMFORT | 1 | 1 | 0 |
| | Disorders and | | | | |
| | administration | | | | |
| | site conditions | | | | |
| | Nervous System | HEADACHE | 0 | 1 | 0 |
| | Disorders | SYNCOPE VASOVAGAL | 0 | 1 | 0 |
| | Respiratory, | COUGH | 3 | 3 | 0 |
| | thoracic and | DYSPNOEA | 1 | 0 | 0 |
| | mediastinal | PLEURITIC PAIN | 1 | 0 | 0 |
| | disorders | | | | |
| Nebu-Tec | General | CHEST DISCOMFORT | 1 | 1 | 0 |
| Optineb | Disorders and | | | | |
| | administration | | | | |
| | site conditions | | | | |
| | Nervous System | DIZZINESS | 0 | 2 | 0 |
| | Disorders | HEADACHE | 0 | 2 | 0 |
| | Respiratory, | COUGH | 4 | 2 | 0 |
| | thoracic and | DRY THROAT | 0 | 1 | 0 |
| | mediastinal | PHARYNGOLARYNGEAL | 0 | 1 | 0 |
| | disorders | PAIN | | | |
| | | PRODUCTIVE COUGHT | 0 | 0 | 1 |

NB: Counts represent the number of subjects experiencing the adverse event within a relationship within each administration.
Output File: tab_ae_rel; Produced: 07JUN2008 14:28; Final

### 12.2.3 Analysis of Adverse Events

A total of 27 treatment emergent adverse events were reported by 9 subjects. Fifteen (15) adverse events were recorded following administration of treprostinil sodium via the AERx Essence System. Twelve (12) adverse events were recorded following administration of treprostinil sodium via the Nebu-Tec Optineb. One (1) adverse event was considered to be unlikely related to the study medication; fifteen (15) adverse events were considered to be possibly related to the study medication and eleven (11) were considered to be probably related to study medication. Twenty-three (23) adverse events were recorded as mild in intensity and four (4) adverse events were recorded as moderate in intensity.

There was one (1) isolated vaso-vegal episode reported for Subject 07 after administration of sodium treprostinil via the AERx Essence System. This occurred 22 minutes after administration of the study medication and lasted for 14 minutes. This adverse event was considered possibly related to study medication and was moderate in intensity.

The most commonly recorded adverse events after administration of treprostinil sodium were follows: cough (productive and non-productive, headache, chest tightness and chest pain, light headedness and dry/sore throat.

### 12.4.1 Listing of Individual Laboratory Measurements by Subject and Each Abnormal Laboratory Value

Clinical laboratory evaluations (Biochemistry, hematology and urinalysis) were performed at screening and at post study assessment. Drugs of abuse, including alcohol assessments were performed at screening and Day −1. The clinical significance of each out of normal range laboratory parameters was determined by the investigator during the study.

### 12.4.2 Evaluation of Each Laboratory Parameter

There were no clinically-significant changes in laboratory parameters observed during the study.

None of the out of range values was considered to be clinically-significant in the opinion of the investigating physician.

### 12.5 Vital Signs, Physical Findings and Other Observations Related to Safety

### 12.5.1 Vital Signs

There were no clinically-significant changes in vital signs (blood pressure, pulse and oral temperature) observed during the study.

On the evening of Day −1 a standing blood pressure was performed as a repeat blood pressure (File note Ref: 17APR08/AJ/03) in order to exclude orthostatic hypotension. None of the out of range values was considered to be clinically-significant in the opinion of the investigating physician.

### 12.5.4 Respiratory Function

All screening results were >80% of predicted value, as required by the protocol for enrolment onto the study.

### 12.5.5 Concomitant Medication

No concomitant medication was taken during the study.

### 12.5.6 Drug/Alcohol and HIV/Hepatitis Screening

All subjects had a negative drugs of abuse result prior to each dose administration.

### 12.6 Safety Conclusions

There were a total of twenty-seven (27) adverse events reported by fourteen (14) subjects during the study. Twelve (12) adverse events were recorded following administration of treprostinil sodium via the Nebu-Tec Optineb. One (1) adverse event was considered to be unlikely related to the study medication; fifteen (15) adverse events were considered to be possibly related to the study medication and eleven (11) were considered to be probably related to study medication. Twenty-three (23) adverse events were recorded as mild in intensity and four (4) adverse events were recorded as moderate in intensity.

There was one (1) isolated vaso-vegal episode reported for Subject 07 after administration of sodium treprostinil via the AERx Essence System. This occurred 22 minutes after administration of the study medication and lasted for 14 minutes. This adverse event was considered possibly related to study medication and was moderate in intensity.

There were no Serious Adverse Events (SAE's) or Suspected Unexpected Serious Adverse Reactions (SUSAR's) reported during the study.

There were no clinically-significant changes in laboratory parameters, physical examination, vital signs, respiratory function or ECGs during the study.

In conclusion, treprostinil sodium for inhalation was considered to be well-tolerated in healthy subjects in this study.

### 13. Discussion and Overall Conclusions

There were no Serious Adverse Events (SAE's) or Suspected Unexpected Serious Adverse Reactions (SUSAR's) reported during the study.

There were no clinically-significant changes in laboratory parameters, physical examination, vital signs, respiratory function or ECGs during the study.

In conclusion, treprostinil sodium for inhalation was considered to be well-tolerated in healthy subjects in this study.

Scintigraphic analysis showed that for both devices the majority of the emitted dose was deposited within the lung (Table 11.4.1.1.1). However, lung deposition from AERx was statistically significantly greater than that from the Optineb device. For both devices the extra pulmonary deposition was low, although it was statistically greater for Optineb (Table 11.4.1.1.4).

Scintigraphic data were used to determine the fraction of the loaded dose delivered to the lung which was subsequently utilised to estimate the lung dose in terms of μg of Treprostinil. For AERx, the calculation of lung dose was derived by normalising the emitted dose to lung for the fraction (%) of the loaded dose retained post-administration i.e. within the device and dosage forms. The actual dose within the AERx strips (a single dose consisted of two strips) was calculated from the nominal treprostinil concentration adjusted for the actual concentration determined by HPLC assay of the stock radiolabeled drug solutions on each dosing day (see Table 14.2.1.5).

One administration from AERx resulted in an unusually high retention of radioactivity in the device post-dose (Subject 004, see Table 14.2.1.1). As a consequence the calculated dose to lung for this subject was lower than that observed for the other subjects. Inspection of the gamma scintigraphy images for this subject confirmed that one of the AERx strips had delaminated during dosing causing the unusually high retention within the device. This event increased the overall variability of AERx performance, however, data for this subject were not excluded from the statistical analysis.

The Optineb device contained a drug reservoir (nebuliser cup) from which each dose (six separate puffs) was metered. To determine the dose available to the subject an in vitro test to collect a single emitted dose (ED) was performed for each device following subject dosing. The amount of drug collected during this test was quantified using HPLC analysis. The ED was corrected for mouthpiece hold up and the dose to lung (see Table 14.2.1.6) was calculated as the product of the % emitted dose in the lung and the ED (corrected for mouthpiece hold up).

Analysis of the pattern of distribution of radiolabel within the lung (sC/P) (Table 11.4.1.1.1) showed that the deposition from AERx was statistically more homogeneous i.e. penetrating into the peripheral airways (Table 11.4.1.1.5), than the predominantly central airways deposition following Optineb dosing.

Analysis of the PK data indicated some statistically significant differences between the two treatments, Cₘₐₓ was lower and Tₘₐₓ longer for AERx compared to Optineb (Table 11.4.1.2.2). Analysis of AUC_{T} and AUC_{I} parameters showed that both were statistically significantly greater following AERx dosing compared to Optineb (Table 11.4.1.2.2).

However, the key PK parameters were also calculated following normalisation for the dose of treprostinil delivered to the lung (analysed in Table 11.4.1.2.3, and listed in Tables 14.2.1.5 and 14.2.1.6). Statistical analysis of the dose normalised Cₘₐₓ indicated that AERx was approximately 60% of that following Optineb (Table 11.4.1.2.4). Statistical analysis of the dose normalised AUC parameters showed that although the dose to lung was greater following AERx administration there was no statistically significant difference between the treatments (Table 11.4.1.2.4).

It may be inferred therefore, that the deposition pattern of treprostinil within the lung influenced its rate of systemic availability (as measured by Cₘₐₓ and Tₘₐₓ) but not the relative extent of absorption (as measured by dose normalised AUC_{T} and AUC_{I}).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

### REFERENCE LIST

1. Lung Rx. Investigator's Brochure: Treprostinil for Inhalation, Version: 3.0, March 2007
2. Lung Rx Clinical Trial Protocol BA.001, “An Open-Label, Randomized, Three-Period Crossover Comparative Pharmacokinetics and Steady-State Absolute Bioavailability Study of Treprostinil Sodium for Inhalation and Administration of Remodulin® by Continuous Intravenous Infusion to Normal Healthy Volunteers”. Draft Study Report, May 2007
3. Notes for Guidance on the Clinical Administration of Radiopharmaceuticals and Use of Sealed Radioactive Sources. Administration of Radioactive Substances Advisory Committee (ARSAC) (March 2006). ARSAC Secretariat, Chilton, Didcot, Oxon. OX11 0RQ.
4. Brusasco V, Crapo R, Viegi G. Standardisation of spirometry. Series ATS/ERS Task Force: Standardisation of Lung Function Testing” Eur Respir J 2005; 26: 319-338
5. Boyd, B., Noymer, P., Liu, K., Okikawa, J., Hasegawa, D., Warren, S., Taylor, G., Ferguson, E., Schuster, J., Farr, S., and Gonda, I. (2004) Effect of Gender and Device Mouthpiece Shape on Bolus Insulin Aerosol Delivery Using the AERx Pulmonary Delivery System. Pharmaceutical Research. 21 (10) 1776-1782.
6. Blanchard, J. D., Cipolla, D., Liu, K., Morishige, R., Mudumba, S., Thipphawong, J., Taylor, G., Warren, S., Radhakrishnan, R., Van Vlasselaer, R., Visor, G. and Starko, K. (2003) Lung Deposition of Interferon Gamma-1b following Inhalation via AERx® System vs. Respirgard II™ Nebulizer Proc. ATS Annual Meeting (Abstract A373), Seattle.
7. Publications of the International Commission on Radiological Protection (ICRP) (1977) Recommendations of the International Commission on Radiological Protection 26.
8. Annals of the International Commission on Radiological Protection (ICRP) Vol 28, No. 3, 1998, Publication 80, Radiation Dose to Patients from Radiopharmaceuticals.
9. National Radiological Protection Board. Doses to Patients from Medical Radiological Examinations in Great Britain. (1986) Radiological Protection Bulletin No. 77.
10. Agnew J E, Bateman R M, Pavia D, Clarke S W. (1984) Radionuclide demonstration of ventilatory abnormalities in mild asthma. Clinical Science; 66: 525-531.
11. Colthorpe P, Taylor G, Farr S J. (1997) A comparison of two non-invasive methods for quantifying aerosol deposition in the lungs of rabbits. J. Aerosol Med.; 10:255

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All the publications, patent applications and patents cited in this specification are incorporated herein by reference in their entirety.

That which is claimed is:

1. A method of treating or preventing a disease or condition, which is treatable or preventable with treprostinil, comprising:
administering by inhalation to a subject in need thereof an aerosolized formulation comprising treprostinil or a pharmaceutically acceptable salt thereof and a carrier acceptable for pulmonary delivery, wherein said aerosolized formulation has an aerodynamic diameter of particles or droplets of no more than 10 microns and wherein said administering results in depositing the treprostinil in a deep lung, such that a ratio of central/peripheral lung deposits of the formulation is in a range of 1 to 2.0.

2. The method of claim 1, wherein the ratio of central lung to peripheral lung deposits of the formulation on lung is 1 to 1.5.

3. The method of claim 1, wherein the ratio of central lung to peripheral lung deposits of the formulation on lung is 1 to 1.45.

4. The method of claim 1, wherein the subject is a human.

5. The method of claim 1, applied for treating pulmonary hypertension.

6. The method of claim 1, wherein the formulation comprises treprostinil sodium.

7. The method of claim 1, wherein said aerodynamic diameter is in a range from 2 microns to 10 microns.

8. The method of claim 1, wherein said aerodynamic diameter is no more than 5 microns.

9. The method of claim 1, wherein said formulation is a liposome-free formulation.
